(19) United States
(12) Patent Application Publication
(10) Pub. No.: US 2015/0203917 A1
(43) Pub. Date: Jul. 23, 2015

(54) PROGNOSIS BIOMARKERS IN CARTILAGE DISORDERS

(71) Applicant: MERCK PATENT GMBH, Darmstadt (DE)

(72) Inventors: Christoph Hubertus Ladel, Darmstadt (DE); Alix Anne Simone Berton, Biberach An Der Riß (DE); Armand Valsesia, Chavannes-Près-Renens (CH); Pierre Jacques Farmer, Evians-Les-Bains (FR)

(21) Appl. No.: 14/420,109

(22) PCT Filed: Aug. 5, 2013
(86) PCT No.: PCT/EP2013/066422

§ 371 Date: Feb. 6, 2015

Related U.S. Application Data
(60) Provisional application No. 61/680,493, filed on Aug. 7, 2012.
(60) Provisional application No. 61/778,950, filed on Mar. 13, 2013.

(30) Foreign Application Priority Data
Aug. 6, 2012 (EP) 12179393.9

(51) Int. Cl. C12Q 1/68

(52) U.S. Cl. CPC C12Q 1/6883; C12Q 2600/16; C12Q 2600/156; C12Q 2600/118; C12Q 2600/112

(57) ABSTRACT

This application is directed to the use of biomarkers for prognosing disease severity in a subject having a cartilage disorder, such as osteoarthritis, cartilage injury, fractures affecting joint cartilage or surgical procedures with impact on joint cartilage (e.g., microfracture). It also describes a method of predicting sensitivity to a drug prior to drug administration in a subject having a cartilage disorder, as well as clinical management based on the likelihood of said patients of being non-sensitive, sensitive or highly sensitive to a drug treatment.

### FIELD OF INVENTION

The present invention relates, generally, to pharmacogenetics, more specifically to genetic markers associated with severity of a cartilage disorder or progression of said cartilage disorder. The present invention more particularly relates to human genes, which can be used for the diagnosis and treatment of cartilage disorders.

The invention further discloses specific polymorphisms or alleles of the URN gene that are related to cartilage response to a drug treatment, such as an anabolic drug, as well as diagnostic tools and kits based on these susceptibility alterations. Thus, the invention can be used in predicting the response to a drug treatment. It could be used for selecting/identifying patients to be treated by intra-articular administration of a given drug compound. The use of these markers in diagnostics could result in increased benefit and reduced risk in patients.

### BACKGROUND OF THE INVENTION

Cartilage disorders broadly refer to diseases characterized by degeneration of metabolic abnormalities in the connective tissues which are manifested by pain, stiffness and limitation of motion of the affected body parts. These disorders can be due to pathology or can be the result of trauma or injury. Among others, cartilage disorders include osteoarthritis (OA) and cartilage injury (inclusive sports injuries of cartilage and joint, or surgical injuries such as microfracture(s)). Mature cartilage has limited ability to repair itself, notably because mature chondrocytes have little potential for proliferation and due to the absence of blood vessels. In addition, cartilage is not well nutrified and has a low oxygen pressure. Replacement of damaged cartilage, in particular articular cartilage, caused either by injury or disease is a major challenge for physicians, and available surgical treatment procedures are considered not completely predictable and effective for only a limited time. Therefore, the majority of younger patients either does not seek treatment or are counseled to postpone treatment for as long as possible. When treatment is required, the standard procedure is age dependent and varies between total joint replacement, transplantation of pieces of cartilage or marrow stimulating technique (such as microfracture). Microfracture is a common procedure that involves penetration of the subchondral bone to stimulate cartilage deposition by bone marrow derived stem cells. However, it has been shown that this technique does not repair sufficiently the chondral defect and the new cartilage formed is mainly fibrocartilage, resulting in inadequate or altered function and biomechanics. Indeed, fibrocartilage does not have the same durability and may not adhere correctly to the surrounding hyaline cartilage. For this reason, the newly synthesized fibrocartilage may breakdown more easily (expected time frame: 5-10 years). For patients with osteoarthritis, non-surgical treatment consists notably of physical therapy, lifestyle modification (e.g. reducing activity), supportive devices, oral and injected drugs (e.g. non-steroidal anti-inflammatory drugs), and medical management. Once these treatments fail, surgery, such as joint replacement, is the main option for the patients. Such an option can provide a reduction in symptoms that are generally only short lived. Tibial or femoral osteotomies (cutting the bone to rebalance joint wear) may reduce symptoms, help to maintain an active lifestyle, and delay the need for total joint replacement. Total joint replacement can provide relief for the symptom of advanced osteoarthritis, but generally requires a change in a patient's lifestyle and/or activity level. At that time, drug treatments on the market are mainly directed to pain relief. There is not yet commercially available treatment that restores the cartilage damages (see Lotz, 2010).

Fibroblast Growth factor 18 (FGF-18) is a member of the FGF family of proteins, closely related to FGF-8 and FGF-17. It has been shown that FGF-18 is a proliferative agent for chondrocytes and osteoblasts (Ellsworth et al., 2002; Shimoaka et al., 2002). FGF-18 has been proposed for the treatment of cartilage disorder such as osteoarthritis and cartilage injury either alone (WO2008/023063) or in combination with hyaluronic acid (WO2004/032849).

Sprifermin, which is a truncated form of human FGF-18, is being investigated in clinical trials for treatment of both osteoarthritis and cartilage injury (for more details see for instance NCT01033994, NCT00911469 and NCT01066871). The current dosing regimen for sprifermine is once weekly for 3 weeks (one treatment cycle), the drug being administered via intraarticular injections. This treatment cycle can be repeated. This dosing regimen has been described in WO2008023063.

At that time, OA and cartilage injury treatments with any drug, notably anabolic drug such as sprifermin, during clinical trials, are provided to patients without predictive information on the response, i.e. without knowledge on whether the treatment will likely be highly effective, moderately effective or show only little or no effect. Currently, for instance, numerous treated patient population exhibit an intermediate/high response to treatment according to the WOMAC scores with FGF18 after at least one treatment cycle, however, some others either do not respond to said treatment or respond while presenting high WOMAC score compared to control.

Here we describe for the first time genetic markers that are associated with the quality of the clinical response to treatment of a cartilage disorder, such as osteoarthritis, cartilage injury, fractures affecting joint cartilage or surgical procedures with impact on joint cartilage (e.g. Microfracture), with a drug, such as FGF18. Such markers are useful for identifying, through genetic screening prior to the treatment subgroups of patients that are more likely to exhibit a particular response to treatment with said drug, such as a very good clinical response to treatment with the drug or on the contrary those for whom the therapy may fail. Knowledge on the type of clinical response of a patient to treatment can be used to optimize therapy or select therapy, such as selecting treatment with a given drug as a first line therapy or adapting the dosing regimen. Such information will be clinically useful for the medical management of cartilage disorders, such as OA/cartilage injury, in patients. For example, if an individual with OA or cartilage injury is known to be at increased risk for not responding to an FGF18 treatment, the physician may exclude said patient from said treatment. Such prognostic information may also be clinically useful to guide decisions on the dosing regimen.

In addition, the present invention describes genetic markers that are prognostic of the disorder severity or of the disorder progression. Such markers are useful for identifying, through genetic screening prior to the treatment, subgroups of patients that are more likely to exhibit less severe form of cartilage disorder. Such prognostic information may thus be clinically useful to guide medical decisions.

### SUMMARY OF THE INVENTION

The present invention is directed to a method of prognosing disorder severity, or disorder progression, in a subject having a cartilage disorder, the method comprising the steps of:
a. Determining, from a nucleic acid sample, the genotype at both IL-1 RN rs9005 and IL-1 RN rs315952;
b. Prognosing from the result of step a disorder severity.

According to said method, the presence of the genotype G/G at IL-1 RN rs9005 and T/T at IL-1 RN rs315952 is predictive of less severe form of cartilage disorder.

Also described herein, a method for predicting sensitivity to a drug prior to drug administration in a subject having a cartilage disorder, the method comprising the steps of:
a. Determining, from a nucleic acid sample, the genotype at both IL-1 RN rs9005 and IL-1 RN rs315952;
b. Predicting from the result of step a. high, intermediate, low or no sensitivity of said subject to a said drug.

According to said method, the presence of the genotype G/G at IL-1 RN rs9005 and T/T at IL-1 RN rs315952 is predictive of low or no sensitivity to the drug. On the contrary, the presence of the genotype(s) selected from the group consisting of (1) IL-1 RN rs9005 G/G and IL-1 RN rs315952 T/C or C/C, and (2) IL-1 RN rs9005 NG or NA and IL-1 RN rs315952 T/T, T/C or C/C is predictive of sensitivity to said drug. In particular, the presence of the genotype NA or NG at IL-1 RN rs9005 and C/C or C/T at IL-1 RN rs315952 is predictive of high sensitivity to said drug.

In a further embodiments, is also herein described a method for selecting a patient having a cartilage disorder for inclusion in or exclusion from treatment, or clinical trial, with a drug, based on the likelihood of his/her sensitivity to said drug, comprising the steps of:
a. Identifying the patient's nucleic acid at both of the polymorphic loci consisting of IL-1 RN rs9005 and IL-1RN rs315952, wherein the patient's genotype with respect to said loci is predictive about the patient's risk for being sensitive or not sensitive to said drug, and
b. Selecting the sensitive patients as being suitable for treatment, or clinical trial, with said drug.

In particular, patients having the genotype IL-1 RN rs9005 G/G and IL-1 RN rs315952 T/T will be classified as non-sensitives. As such, these subjects could be excluded from the drug treatment, or from clinical trial. It follows that the subjects having any other genotypes at these loci (i.e. IL-1 RN rs9005 G/G and IL-1 RN rs315952 T/C or C/C or IL-1 RN rs9005 NG or NA and IL-1 RN rs315952 T/T, T/C or C/C) will be classified as sensitives, comprising both intermediate-sensitives and super-sensitives (or high-sensitives) subjects, and thus could be included in treatment with the therapeutic compound.

The present invention further provides a method for selecting patients having a cartilage disorder for an alternative therapeutic regimen with a drug, based on their likelihood of being super-sensitives to said drug, comprising identifying the patient's nucleic acid at both of the polymorphic loci selected from the group consisting of IL-1RN rs9005 and IL-1RN rs315952, wherein the patient's genotype with respect to said loci is predictive about the subject's risk for being super-sensitive to a treatment with said drug and selecting said patient for an alternative therapeutic regimen that would be suitable to said patient. Preferably, in such alternative therapeutic regimen, the total dose of drug that is to be administered could be reduced compared to the dose of said drug to be administered to a patient who does not present a risk for being super-sensitive. In particular, patients having the genotype IL-1 RN rs9005 NG or A/A together with IL-1 RN rs315952 T/C or C/C, being classified as super-sensitives, are selected for an alternative therapeutic regimen in which one the dose of the drug to be administered is reduced.

The present invention also provides a method for selecting patients having a cartilage disorder for an alternative therapeutic regimen with a drug, based on their likelihood of having AIR events when treated with said drug, comprising identifying the patient's nucleic acid at both of the polymorphic loci selected from the group consisting of IL-1RN rs9005 and IL-1RN rs315952, wherein the patient's genotype with respect to said loci is predictive about the subject's risk for developing AIR events in response to treatment with said drug and selecting said patient for an alternative therapeutic regimen that would be suitable to said patient. Preferably, in such alternative therapeutic regimen, the total dose of drug that is to be administered could be reduced compared to the dose of said drug to be administered to a patient who does not present a risk for developing AIR events. In particular, patients having the genotype IL-1 RN rs9005 A/G or NA together with IL-1RN rs315952 T/C or C/C, being classified as super-sensitives, are selected for an alternative therapeutic regimen in which one the dose of the drug to be administered is reduced.

In a further aspect, it is also described a kit comprising means for performing the above methods and instructions for use. Said kit includes at least a couple of specific primers or probes for detecting the presence or absence of the alleles.

In the context of the present invention as a whole, the patient has a cartilage disorder preferably selected from the group consisting of osteoarthritis, cartilage injury and fractures affecting joint cartilage or surgical procedures with impact on joint cartilage (e.g. Microfracture).

In a particular embodiment of the present invention as a whole, the drug to be administered is a drug that is administered in order to treat a cartilage disorder. In a preferred embodiment, said drug has an anabolic effect on cartilage. Among said anabolic drugs, are included: BMP-2, BMP-7, GDF-5, FGFβ, FGF-8, FGF-9, SOX-9 enhancers, TGFβ, and any variants thereof, as well as FGF-18 compounds, such as sprifermin.

It is to be understood that in any of the methods or uses mentioned herein, before determining the genotype at one locus, it is needed to obtain a nucleic acid sample (or a test sample) of said subject, for instance by blood or saliva collecting. Alternatively the test sample is selected from bucall cells, urine or stool. Preferably, the nucleic acid sample is a DNA sample. Further, it is also to be understood that any of the methods or uses mentioned herein are performed in vitro, and not on the animal or human body.

### DEFINITIONS

The term “drug” or “therapeutic compound” means a compound that is currently used or that could be used in therapy of a cartilage disorder.
The term “anabolic compound” or “anabolic drug” is to be understood as a compound or a drug that has anabolic effect on the cartilage, preferably leading to cartilage repair. Among such compound can be listed FGF-18 compound (as defined herein), BMP-2 (e.g. reference Uniprot P12643), BMP-7 (e.g. reference Uniprot P18075), GDF-5 (e.g. reference Uniprot P43026), FGFβ (e.g. reference Uniprot P09038), FGF-8 (e.g. reference Uniprot P55075), FGF-9 (e.g. reference Uniprot P31371), SOX-9 enhancers (e.g. reference Uniprot P48436 for SOX-9) or TGFβ (e.g. reference Uniprot P01137) and any variants thereof.
The term “FGF-18 compound” or “FGF-18”, as used herein, is intended to be a protein maintaining at least one biological activity of the human FGF-18 protein. FGF-18 may be native, in its mature form, or a truncated form thereof. Biological activities of the human FGF-18 protein include notably the increase in osteoblastic activity (see WO98/16644) or in cartilage formation (see WO2008/023063). Native, or wild-type, human FGF-18 is a protein expressed by chondrocytes of articular cartilage. Human FGF-18 was first designated zFGF-5 and is fully described in WO98/16644. SEQ ID NO:1 corresponds to the amino acid sequence of the native human FGF-18, with a signal peptide consisting of amino acid residues 1 (Met) to 27 (Ala). The mature form of human FGF-18 corresponds to the amino acid sequence from residue 28 (Glu) to residue 207 (Ala) of SEQ ID NO: 1 (180 amino acids). The term also includes fusion protein, wherein FGF-18 protein is coupled with a heterologous protein or a chemical compound.

FGF-18, in the present invention, may be produced by recombinant methods, such as taught by the application WO2006/063362. Depending on the expression systems and conditions, FGF-18 in the present invention is expressed in a recombinant host cell with a starting Methionine (Met) residue or with a signal sequence for secretion. When expressed in prokaryotic host, such as in E. coli, FGF-18 contains an additional Met residue in N-terminal of its sequence. For instance, the amino acid sequence of human FGF-18, when expressed in E. coli, starts with a Met residue in N-term (position 1) followed by residues 28 (Glu) to residue 207 (Ala) of SEQ ID NO: 1.
The term “truncated form” of FGF18, as used herein, refers to a protein which comprises or consists of residues 28 (Glu) to 196 (Lys) of SEQ ID NO: 1. Preferably, the truncated form of FGF-18 protein is the polypeptide designated “trFGF-18” (170 amino acids), which starts with a Met residue (in N-terminal) followed by amino acid residues 28 (Glu)-196 (Lys) of the wild-type human FGF-18. The amino acid sequence of trFGF-18 is shown in SEQ ID NO:2 (amino acid residues 2 to 170 of SEQ ID NO:2 correspond to amino acid residues 28 to 196 of SEQ ID NO:1). trFGF-18 is a recombinant truncated form of human FGF-18, produced in E. coli (see WO2006/063362). The International Nonproprietary Name (INN) for this particular form of FGF-18 is sprifermin. Sprifermin has been shown to display similar activities as the mature human FGF-18, e.g. it increases chondrocyte proliferation and cartilage deposition leading to repair and reconstruction for a variety of cartilaginous tissues (see WO2008/023063).
“Cartilage disorder”, as used herein, encompasses disorders resulting from damages due to injury, such as traumatic injury, chondropathy or arthritis. Examples of cartilage disorders that may be treated by the administration of the FGF-18 formulation described herein include, but are not restricted to, arthritis, such as osteoarthritis, cartilage injury, fractures affecting joint cartilage or surgical procedures with impact on joint cartilage (e.g. Microfracture). Degenerative diseases/disorders of the cartilage or of the joint, such as chondrocalcinosis, polychondritis, relapsing polychondritis, ankylosing spondylitis or costochondritis are also encompassed by this wording. The International Cartilage Repair Society has proposed an arthroscopic grading system to assess the severity of the cartilage defect: grade 0: (normal) healthy cartilage, grade 1: the cartilage has a soft spot or blisters, grade 2: minor tears visible in the cartilage, grade 3: lesions have deep crevices (more than 50% of cartilage layer) and grade 4: the cartilage tear exposes the underlying (subchronal) bone (see for instance page 13 of http://www.cartilage.org/_files/contentmanagement/ICRS_evaluation.pdf).
The term “Osteoarthritis” is used to intend the most common form of arthritis. The term “osteoarthritis” encompasses both primary osteoarthritis and secondary osteoarthritis (see for instance The Merck Manual, 17^{th} edition, page 449). The most common way of classifying/grading osteoarthritis is the use of the Kellgren-Lawrence radiographic grading scale (see table below). Osteoarthritis may be caused by the breakdown of cartilage. Bits of cartilage may break off and cause pain and swelling in the joint between bones. Over time, the cartilage may wear away entirely, and the bones will rub together. Osteoarthritis can affect any joint but usually concerns hands and weight-bearing joints such as hips, knees, feet, and spine. In a preferred example, the osteoarthritis may be knee osteoarthritis or hip osteoarthritis. Osteoarthritis is one of the preferred cartilage disorders that can be treated by administering the FGF-18 compounds according to the present invention.

Kellgren-Lawrence Radiographic Grading Scale of Osteoarthritis is described as follow:

| Grade of | |
|---|---|
| Osteoarthritis | Description |
| 0-None | No radiographic findings of osteoarthritis |
| 1-Doubtful | Doubtful narrowing of joint space and possible |
| | osteophytic lipping |
| 2-Minimal | Definite osteophytes, definite narrowing of joint space |
| 3-Moderate | Moderate multiple osteophytes, definite narrowing of |
| | joints space, some sclerosis and possible deformity of |
| | bone contour |
| 4-Severe | Large osteophytes, marked narrowing of joint space, |
| | severe sclerosis and definite deformity of bone contour |

The term “cartilage injury” as used herein is a cartilage disorder or cartilage damage resulting notably from a trauma. Cartilage injuries can occur notably after traumatic mechanical destruction, notably further to an accident or surgery (for instance microfracture surgery). This term “cartilage injury” also includes chondral or osteochondral fracture, damage to meniscus, and the term microfracture. Also considered within this definition is sport-related injury or sport-related wear of tissues of the joint.
The term “disease severity” is related to the grade of the cartilage disorder: the higher, the more severe. For instance, a grade 3 according to Kellgren-Lawrence grading system is more severe than a grade 2 according to the same grading system.
The term AIR (acute inflammatory reaction) as used herein is defined as follow. Within 1 to 7 day-period, preferably within 3 day-period, following the intra-articular injection of FGF-18 compound in the target knee both, the following criteria must be fulfilled:
   Self-reported swelling (synovial fluid effusion)
   Pain increase by 30 mm on 100 mm Visual Analogue Scale (VAS)
An “allele” is a particular form of a gene, genetic marker or other genetic locus, that is distinguishable from other forms of the gene, genetic marker or other genetic locus; e.g. without limitation by its particular nucleotide sequence. The term allele also includes for example without limitation one form of a single nucleotide polymorphism (SNP). An individual can be homozygous for a certain allele in diploid cells; i.e. the allele on both paired chromosomes is identical; or heterozygous for said allele; i.e. the alleles on both paired chromosomes are not identical.
The term a “genetic marker”, “biomarker” or “marker” refers to an identifiable polymorphic (genetic) locus. An example without limitation of a genetic marker is a single nucleotide polymorphism (SNP).
A “single nucleotide polymorphism (SNP)” is a DNA sequence variation occurring when a single nucleotide—A (for Adenine), T (for Thymine), C (for Cytosine), or G (for Guanine)—in the genome (or other sequence shared between individuals of a species) differs between individuals of a species (or between paired chromosomes in an individual). A SNP is frequently preceded by and followed by highly conserved sequences in the population of interest and thus the location of a SNP is typically made in reference to a consensus nucleic acid sequence of thirty to sixty nucleotides that bracket the genetic marker locus, which is sometimes referred to as a context sequence for the SNP. The SNPs that were analyzed by the present inventors in connection with treatment of cartilage disorder with sprifermin are those shown in Table 1.
A “genotype” as used herein refers to the combination of both alleles of a genetic marker, e.g. without limitation of a SNP, on a single genetic locus on paired (homologous) chromosomes in an individual. “Genotype” as used herein also refers to the combination of alleles of more than one genetic loci, e.g. without limitation of SNPs, on a pair or more than one pair of homologous chromosomes in an individual.
The term “Haplotype” refers to variants or alleles from distinct markers (e.g. SNPs) that are co-located on the same chromosome. SNP genotype data, as measured from SNP arrays or Taqman assays, are unphased (i.e. the chromosome's parent of origin is unknown for each allele). Computational methods (Browning et Browning, 2011) use information across individuals to estimate (i.e. infer) haplotype phase from genotype data.
The term “Genotyping” refers to a process for determining a genotype of an individual, either for a single SNP or many SNPs.
“Locus” or “genetic locus” refers to a specific location on a chromosome or other genetic material. For instance, IL-1 RN rs9005 is a locus and can be called, in the frame of the present invention, either “IL-1 RN rs9005” or “locus IL-1 RN rs9005”. The same applies to IL-1 RN rs315952. As self evident for the skilled person, from NCBI database for these SNPs, the genotype to be determined at both IL-1 RN rs9005 and IL-1 RN rs315952, is the one in position 27 of each of these loci, i.e. position 27 of SEQ ID NO:6 and position 27 of SEQ ID NO:7.
The term “SNP1” in the context of the present invention, is position 27 of SEQ ID NO: 6, also identified as rs9005 in NCBI database. SEQ ID NO. 6 is a portion of genomic nucleic acid sequence of interleukin 1 receptor antagonist (IL-1 RN). The terms “IL-1 RN rs9005”, “rs9005” or “SNP1” are used interchangeably.
The term “SNP2” refers to position 27 of SEQ ID NO. 7 identified as being rs315952 in NCBI database. SEQ ID NO. 7 is a portion of genomic nucleic acid sequence of IL-1 RN. The terms “IL-1RN rs315952”, “rs315952” or “SNP2” are used interchangeably.
The term “probe” or “primer” refers to an oligonucleotide, i.e. a nucleic acid or a nucleic acid derivative; including without limitation a locked nucleic acid (LNA), peptide nucleic acid (PNA) or bridged nucleic acid (BNA); that is usually between 5 and 100 contiguous bases in length, and most frequently between 5-40, 5-35, 5-30, 5-25, 5-20, 5-15, 5-10, 10-50, 10-40, 10-30, 10-25, 10-20, 15-50, 15-40, 15-30, 15-25, 15-20, 20-50, 20-40, 20-30 or 20-25 contiguous bases in length. The sequence of a probe/a primer can be designed to specifically hybridize to one of the allelic forms of a genetic marker; such oligonucleotides are referred to as allele-specific probes. If the genetic marker is a SNP, the complementary allele for that SNP can occur at any position within an allele-specific probe. Other probes/primers useful in practicing the invention specifically hybridize to a target region adjacent to a SNP with their 3′ terminus located one to less than or equal to about 10 nucleotides from the genetic marker locus, preferably ≦ about 5 nucleotides. Such probes/primers hybridizing adjacent to a SNP are useful in polymerase-mediated primer extension methods and are referred to herein as “primer-extension oligonucleotides.” In a preferred embodiment, the 3′-terminus of a primer-extension oligonucleotide is a deoxynucleotide complementary to the nucleotide located immediately adjacent a SNP.
The term “Polymorphism” refers of two or more alternate forms (alleles) in a population of a genetic locus that differ in nucleotide sequence or have variable numbers of repeated nucleotide units. Polymorphisms occur in coding regions (exons), non-coding regions of genes or outside of genes (intergenic regions). The different alleles of a polymorphism typically occur in a population at different frequencies, with the allele occurring most frequently in a selected population sometimes referenced as the “major” or “wild type” allele. Diploid organisms may be homozygous or heterozygous for the different alleles that exist. A biallelic polymorphism has two alleles.
The term “Epistasis” is generally used to define the interaction between genes. Epistasis was first defined by Bateson (Bateson et Mendel, 1909) to describe a masking effect whereby a variant or allele at one locus prevents the variant at another locus from manifesting its effect. However the scientific literature provides many different definitions (Phillips, 1998; Cordell, 2002). Herein, epistasis was tested as the statistical interaction between genotypes from two distinct SNPs. This is similar to the definition proposed by Fisher in 1918 (Fisher, 1918), i.e. a deviation from additivity in the effect of alleles at different loci with respect to their contribution to a phenotype.
“WOMAC total scores” or “WOMAC scores” (“WOMAC” for “Western Ontario and McMaster Universities Osteoarthritis Index”) measure pain (WOMAC pain score), function (WOMAC function score) and stiffness (WOMAC stiffness score). When applied to assessing pain and dysfunction associated with cartilage injury, it consists of a questionary containing 24 items divided into 3 subscales (5 items for Pain, 2 items for Stiffness and 17 items for Physical Function) (see Bellamy et al., 1988; Wolfe, 1999). It is a well-known instrument, widely used notably in assessment of the OA severity.
Cartilage volume measurements were performed through magnetic resonance imaging (MRI) measurements, including Total volume of cartilage (LFTC+MFTC), Lateral volume of cartilage (also referred as LFTC: lateral femoro-tibial compartment), Medial volume of cartilage (also referred as MFTC: medial femoro-tibial compartment), and new total average cartilage thickness.
The term “baseline” means before treatment (i.e. at study entry). It refers notably to clinical variables, such as, but not limited to, the cartilage volume and WOMAC total score of one given patient at study entry (i.e. before treatment with FGF-18 compound or placebo).
“Sensitives” are patients that exhibit a response to treatment of a cartilage disorder with a drug, preferably an anabolic drug, such as FGF-18. Preferably, sensitive patients (or patients showing sensitivity to treatment) exhibit notably a higher increase in total cartilage volume than placebo treated subjects, i.e. they show cartilage repair. In addition, sensitive patients exhibit at least similar improvement in WOMAC total scores than placebos. The terms “Super-sensitives”, “intermediate-sensitives” and “Non-sensitives” refer to the different groups of patients depending notably on the increase of the cartilage volume following drug treatment. Super-sensitive display a high response (i.e. high cartilage repair) to treatment with said drug, intermediate-sensitive display a good or intermediate response (i.e. good or intermediate cartilage repair) to treatment with said drug, and non-sensitives display no or low response to treatment with said drug. Both super-sensitive and sensitive subjects have similar improvement in WOMAC total score than placebos. Conversely non-responders have significantly smaller improvement in WOMAC total score than placebos. The term “super-sensitives” or “high-sensitives” are used interchangeably. It is noted that super-sensitives have been shown to present higher risk of AIR events.

More particularly, the terms “Intermediate-sensitives”, “Super-sensitives”, and “Non-sensitives” include, but are not limited to, the different groups of patients depending on the increase of the cartilage volume and improvement of WOMAC total score, following drug treatment.

The proposed criteria for sensitives are the following:
1. Positive cartilage increase (between +10 and +100 mm³) compared to baseline,
2. Cartilage increase change significantly higher than change in placebo (e.g. as tested with a linear model adjusting for BMI, KL grade, sex and age and with alpha=5%),
3. WOMAC score improvement, i.e. diminution, (e.g. more than 5 points reduction) compared to baseline,
4. WOMAC score change not significantly higher than change in placebo (e.g. as tested with a linear model adjusting for BMI, KL grade, sex and age and with alpha=5%).

The proposed criteria for super-sensitives are the same than for sensitives, but with cartilage increase greater than 100 mm³ (criterion #1) compared to baseline.

Non-sensitives can be defined as subjects not fulfilling criteria #1 or #2 and not fulfilling criteria #3 or #4.

Intermediate sensitives display a good or intermediate response (or a good or intermediate sensitivity) to treatment with said drug (see above criteria; for instance, median change when treated with an FGF-18 compound: +84.81 mm³ total cartilage volume increases compared to baseline; median change: −20 points on the WOMAC total score compared to baseline; and non-significant difference in WOMAC total score compared to placebos). Super-sensitives display a high response to treatment with said drug (see above criteria; for instance, median change when treated with an FGF-18 compound: +119.46 mm³ total cartilage volume increase compared to baseline, representing a+40.85% increase (i.e. benefit) compared to sensitive subjects, median change: −10 points on the WOMAC total score, both compared to baseline, and non-significant difference in WOMAC total score compared to placebos). Non-sensitives display no or low response to treatment with said drug (see above criteria; for instance, median change when treated with an FGF-18 compound: significantly smaller increase in total cartilage volume compared to placebos (difference between medians: −106.64 mm³); total cartilage; little improvement (median change: −1 point) in WOMAC total scores compared to baseline, and significant difference in WOMAC total score compared to placebos).
The “response”, or “sensitivity” to a drug treatment is to be understood as 1 year after the first injection and measured as 1) increase of cartilage volume, measured owing to MRI or X-Ray for instance, 2) decrease of WOMAC total scores, and 3) changes in WOMAC total scores not significantly higher than those from placebos (refer also to the definition of “sensitive”).
A “prognostic biomarker” is informative about the subject condition, including and not limited to disease evolution, disease severity or disease outcome, regardless of any therapy. A “predictive biomarker” is informative about the effect of a received therapy, including and not limited to efficacy and safety outcome. The prognostic and predictive definitions are not mutually exclusive thus a biomarker can be both prognostic and predictive.
As used in the present invention, the term “MAD” means Multiple Ascending Dose. When this acronym is followed by a figure, the figure corresponds to the dose at which the drug has been injected during treatment. For instance MAD100 refers to a treatment during which a patient received 100 mcg of said drug (e.g. FGF-18 compound) per injection. The acronym “PL” (and “MADPL”) refers to placebo.
The term “storage device”, as used herein, is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media.
As used herein, the term “stored” refers to a process for encoding information on the storage device. Those skilled in the art can readily adopt any of the presently known methods for recording information on known media to generate manufactures comprising expression level information.

### DETAILED DESCRIPTION OF THE INVENTION

There is a need to prognosing disease severity, or disease progression, in a subject having a cartilage disorder. Biomarkers that predict disease susceptibility, disease severity, or disease progression, are therefore important. These biomarkers are typically referred as prognostic biomarkers and are independent of treatment received by patients. Prognostic biomarkers can also be predictive of the response to drug treatment and conversely predictive biomarkers can also be prognostic.

To optimize the treatment of said patients, it is important to identify biomarkers that could be used as predictors of the disease severity. Said predictors could also be useful to identify high-risk groups either being non-sensitives or on the contrary super-sensitives to a drug treatment. For instance, if one patient having osteoarthritis is known to be at high risk for non-responding to a given drug, preferably an anabolic drug, the physician may decide not to propose said drug to said patient. On the contrary, if one patient having osteoarthritis is known to be at high risk for being super-sensitive to the drug treatment, the physician may decide to adapt the dose regimen, in order to lower the dose of said drug to be administered to said patient. Such predictive information may be clinically useful to guide decisions, and notably on the timing of joint replacement surgery if needed.

The surprising finding of the present invention is based on a study aimed at identifying potential biomarkers associated with disease severity, as well as with the risk of being sensitive or non-sensitive to a drug treatment, preferably an anabolic drug such as sprifermin. The biomarkers used in this study were composed of both candidate genetic markers (see Table 1) and less than 1 million SNPs covering the human genome with a median marker spacing of 680 bases. The association between genetic markers and disease severity or clinical response variables was assessed. The rationale behind this type of analysis was to identify biomarkers that could be prognostic of disease severity, in a patient having a cartilage disorder or predictive of the clinical outcome, for a patient to be treated with a drug, preferably an anabolic drug. These SNPs could be used to stratify and target specific patient populations.

The inventors have surprisingly found an association with certain biomarkers (or SNPs) and disease severity or outcome as well adverse effects of drug therapy. Of special interest are the SNPs IL-1 RN rs9005 and rs315952, both located in the URN gene (see FIG. 1).

These biomarkers have been described in the literature, as being possibly related to disease severity and progression in OA patients (see for instance WO2009/135218 or Attur et al., 2010), using a haplotype (so-called C-T-A haplotype) that includes rs419598 (C), rs315952 (T) and rs9005 (A). WO2009/135218 also discloses that subject having at least the genotype (or pair of alleles) G/G at IL-1RN rs9005 and/or T/T or C/C at IL-1RN rs315952 may be predisposed to severe disease progression, whereas subject having at least the genotype T/T at IL-1 RN rs315952 and/or NA or G/A at IL-1 RN rs9005 may be protected from progression to severe disease. Interestingly, although the literature shows that the C-T-A haplotype is needed to possibly predict the risk of having and developing a severe disease, it is a finding of the present invention that only two of these biomarkers, i.e. rs9005 and rs315952, are sufficient to identify patients having less risk of having and developing a severe form of OA, but are also strongly correlated with responsiveness to a drug treatment, such as an anabolic drug. They are not only sufficient to identify such patients, but also more efficient for said prognosis, than the C-T-A haplotype. The third SNP, i.e. rs419598, does not appear being further involved in the observed phenotype, although described, in the prior art, as being linked to the two other SNPs. Another difference between the C-T-A haplotype and the combination from rs9005 and rs315952, is that the former corresponds to additive effects from allele located on the same phase (same inherited chromosome) while the latter corresponds to epistatic effects (i.e. interaction between alleles from either the same phase or different phase).

In particular, it was found that a genotype T/T of the biomarker rs315952 together with G/G of the biomarker rs9005 is associated with a less severe form of osteoarthritis osteoarthritis (i.e. less severe OA form at baseline and less severe OA development). Subjects from a placebo group, bearing this genotype combination, have significantly higher cartilage growth and significantly higher improvement in WOMAC scores compared to placebos from other genotype combinations. Additional analyses, at baseline, demonstrated that subjects (from any dose regimen during treatment with sprifermin, including placebos) bearing this IL-1RN rs9005 G/G and IL-1RN rs315259 T/T genotype were protected against being classified as Kellgren-Lawrence grade 3, thus protected as having a more advanced/severe osteoarthritis condition.

The present invention is thus directed to a method of prognosing disorder severity, or disorder progression, in a subject having a cartilage disorder, the method comprising the steps of:
a. Determining, from a nucleic acid sample, the genotype at both loci IL-1 RN rs9005 and IL-1 RN rs315952;
b. Prognosing from the result of step a disorder severity, or disorder progression.

Before determining the genotype at one locus, it is needed to obtain a nucleic acid sample of said subject, for instance by blood or saliva collecting. Preferably, the nucleic acid sample is a DNA sample. Thus, the present invention is directed to a method of prognosing disorder severity, or disorder progression, in a subject having a cartilage disorder, the method comprising the steps of:
a. Obtaining a nucleic acid sample of said subject
b. Determining, from said a nucleic acid sample, the genotype at both loci IL-1 RN rs9005 and IL-1 RN rs315952;
c. Prognosing from the result of step b disorder severity, or disorder progression.

According to said method, the presence of the genotype G/G at IL-1 RN rs9005 and T/T at IL-1 RN rs315952 is predictive of a less severe form of cartilage disorder. It follows that patients having this genotype will also be predisposed to less severe disease/disorder progression.

The present invention is also directed to a method of determining disorder severity, or disorder progression, in a human subject having a cartilage disorder, the method comprising the steps of: (a) subjecting a test sample from said subject, diagnosed as having a cartilage disorder, to at least one genotyping assay that determines the genotypes of at least two loci, wherein said at least two loci are: (i) SNP1 and (ii) SNP2, (b) determining the genotypes of said at least two loci; (c) determining from the result of steps (a) and (b) disorder severity, or disorder progression for said subject.

According to said method, the presence of the genotype G/G at IL-1 RN rs9005 (SNP1) and T/T at IL-1 RN rs315952 (SNP2) is predictive of a less severe form of cartilage disorder. It follows that patients having this genotype will also be predisposed to less severe disease/disorder progression. The present invention also relates to an assay to determine disorder severity, or disorder progression, in a human subject having a cartilage disorder, the assay comprising: (a) subjecting a test sample from said human subject, diagnosed as having a cartilage disorder, to at least one genotyping assay that determines the genotypes of at least two loci, wherein said at least two loci are: (i) SNP1 and (ii) SNP2, (b) determining the genotypes of said at least two loci; (c) determining from the result of steps (a) and (b) disorder severity, or disorder progression for said subject. According to said method, the presence of the genotype G/G at IL-1 RN rs9005 (SNP1) and T/T at IL-1 RN rs315952 (SNP2) is predictive of a less severe form of cartilage disorder. It follows that patients having this genotype will also be predisposed to less severe disease/disorder progression. Surprisingly, it has also been found by the present inventors that the alleles “A” of the biomarker rs9005 together with “C” of the biomarker rs315952 are associated with a better response to treatment with a drug, preferably an anabolic drug, such as sprifermin, in subjects afflicted with cartilage disorder. These subjects are called super-sensitives or high-sensitives.

On the contrary, it has also surprisingly been found by the present inventors that the genotype rs315952 T/T together with rs9005 G/G is associated with an absence of, or low, response to treatment with a drug, preferably an anabolic drug, such as sprifermin, in subjects afflicted with cartilage injury. These subjects are called non-sensitives.

Therefore, it is a finding of the present invention that polymorphic loci IL-1 RN rs9005 and IL-1 RN rs315952 can be used in combination as predictive biomarkers of responsiveness of one subject to a drug treatment. In a particular embodiment, the subject will be predicted to be non-sensitive to the drug treatment if he has the genotype IL-1 RN rs9005 G/G together with IL-1 RN rs315952 T/T. On the contrary, the subject will be predicted to be super-sensitive (or a high-sensitive) to the drug treatment if he has the genotype IL-1 RN rs9005 A/G or NA together with IL-1 RN rs315952 T/C or C/C. In any other case (i.e. G/G at rs9005 together with T/C or C/C at rs315952 or NG or NA at rs9005 and T/T at rs315952), the patient will be predicted to be sensitive (or intermediate-sensitive) to the drug treatment.

These two biomarkers rs315952 and rs9005 (also called bi-markers when in combination) are therefore not only prognostic but more important predictive of the response of the patients to a drug treatment.

Also described herein is a method of predicting sensitivity to a drug prior to drug administration in a subject having a cartilage disorder, the method comprising the steps of:
a. Determining, from a nucleic acid sample, the genotype at both loci IL-1 RN rs9005 and IL-1 RN rs315952; and
b. Predicting from the result of step a high, intermediate, low or no sensitivity of said subject to said drug.

Before determining the genotype at one locus, it is needed to obtain a nucleic acid sample of said subject, for instance by blood or saliva collecting. Preferably, the nucleic acid sample is a DNA sample. Thus, the present invention is directed to a method of predicting sensitivity to a drug prior to drug administration in a subject having a cartilage disorder, the method comprising the steps of:
a. Obtaining a nucleic acid sample of said subject
b. Determining, from said a nucleic acid sample, the genotype at both loci IL-1 RN rs9005 and IL-1 RN rs315952;
c. Predicting from the result of step b high, intermediate, low or no sensitivity of said subject to said drug.

According to said method, the presence of the genotype G/G at IL-1 RN rs9005 and T/T at IL-1 RN rs315952 is predictive of low or no sensitivity to said drug. The patient will thus be predicted to be non-sensitive. On the contrary, the presence of the genotype(s) selected from the group consisting of (1) IL-1RN rs9005 G/G and IL-1RN rs315952 T/C or C/C, and (2) IL-1RN rs9005 A/G or A/A and IL-1 RN rs315952 T/T, T/C or C/C is predictive of sensitivity to said drug. In particular, the presence of the genotype NA or NG at IL-1 RN rs9005 and C/C or C/T at IL-1 RN rs315952; is predictive of high sensitivity (high response) to said drug. These patients will thus be predicted to be super-sensitive. It follows from said prediction, that the doctor can easily select only those patients that are predicted to be sensitives, including super-sensitives.

The present invention also relates to an assay to determine sensitivity to a drug treatment or to determine a treatment regimen with a drug treatment, the assay comprising: (a) subjecting a test sample from a human subject, diagnosed as having a cartilage disorder, to at least one genotyping assay that determines the genotypes of at least two loci, wherein said at least two loci are: (i) SNP1 and (ii) SNP2, (b) determining the genotypes of said at least two loci; (c) selecting a patient as being sensitive to a treatment with said drug when at least one of the following combinations of SNPs is determined to be present: (i) SNP1 genotype G/G, or C/C in the complement of the SEQ ID NO: 6; and SNP2 genotype T/C or CC, or A/G or GG in the complement of the SEQ ID NO: 7; or (ii) SNP1 genotype NG or AA, or T/C or T/T/ in the complement of the SEQ ID NO: 6; and SNP2 genotype T/T, or NA in the complement of the SEQ ID NO: 7, or (iii) SNP1 genotype NG or NA, or T/C or T/T in the complement of SEQ ID NO:6 and SNP2 genotype T/C or C/C, or NG or G/G in the complement of SEQ ID No:7 and (d) optionally treating the patient selected in step (c) with said drug.

When the above assay is performed to determine a treatment regimen with a drug, step (c) is optional, whereas step (d) is preferably performed, or is performed.

The present invention further relates to an assay to determine non-sensitivity to a drug treatment, the assay comprising: (a) subjecting a test sample from a human subject, diagnosed as having a cartilage disorder, to at least one genotyping assay that determines the genotypes of at least two loci, wherein said at least two loci are: (i) SNP1 and (ii) SNP2, (b) determining the genotypes of said at least two loci; (c) selecting a patient as being non-sensitive to a treatment with said drug when the following combinations of SNPs is determined to be present: SNP1 genotype G/G, or C/C in the complement of the SEQ ID NO: 6; and SNP2 genotype T/T, or NA in the complement of the SEQ ID NO: 7, and (d) optionally treating the patient selected in step (c) with a therapeutic compound other than said drug.

Before determining the genotype at one locus, in the above disclosed assays, it is needed to obtain a nucleic acid (or test) sample of said subject, for instance by blood or saliva collecting.

The present application also encompasses a method for selecting patients having a cartilage disorder for inclusion in or exclusion from treatment, or clinical trial, with a drug, based on the likelihood of their response to said treatment, comprising:
a. Determining, from a nucleic acid sample, the genotype at both loci IL-1 RN rs9005 and IL-1 RN rs315952, wherein the patient's genotype with respect to said loci is predictive about the patient's risk for being sensitive or non-sensitive to said drug, and
b. Selecting the sensitive patients as being suitable for said treatment or clinical trial.

Before determining the genotype at one locus, it is needed to obtain a nucleic acid sample of said subject, for instance by blood or saliva collecting. Preferably, the nucleic acid sample is a DNA sample. Thus, the present invention is directed to a present application encompasses a method for selecting patients a cartilage disorder for inclusion in or exclusion from treatment, or clinical trial, with a drug, based on the likelihood of their response to said treatment, comprising:
a. Obtaining a nucleic acid sample of said subject,
b. Identifying, from said nucleic acid sample, the patient's nucleic acid at both of the polymorphic loci consisting of IL-1RN rs9005 and IL-1RN rs315952, wherein the patient's genotype with respect to said loci is predictive about the patient's risk for being sensitive or non-sensitive to said treatment, and
c. Selecting the sensitive patients as being suitable for said treatment or clinical trial.

According to said method, patients having the genotype IL-1 RN rs9005 G/G and IL-1 RN rs315952 T/T, who are predicted being non-sensitives, are preferably excluded from the drug treatment or from clinical trial. The others patients (the sensitive patients, including super-sensitive), can be selected as suitable for the treatment, or clinical trial, and thus can be treated with the drug. Alternatively, the method for selecting a patient having a cartilage disorder for inclusion in or exclusion from treatment or clinical trial with a drug based on the likelihood of the patient's sensitivity to said drug, comprised the steps of: (a) subjecting a test sample from a human subject, who is diagnosed as having cartilage disorder, to at least one genotyping assay adapted to determine the genotypes of at least two loci, wherein said at least two loci are: (i) SNP1 SNP2, wherein SNP2 is position 27 of SEQ ID NO. 7 identified by rs315952, wherein the SEQ ID NO. 7 is a portion of genomic nucleic acid sequence of interleukin 1 receptor antagonist (IL-1 RN); and (b) detecting from the genotypes of said at least two loci the presence of a genotype combination selected from: (i) SNP1 genotype G/G, or C/C in the complement of the SEQ ID NO: 6; and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or A/A in the complement of the SEQ ID NO: 7; or (iii) SNP1 genotype G/G, or C/C in the complement of the SEQ ID NO: 6 and SNP2 genotype T/T, or NA in the complement of the SEQ ID NO: 7; and (c) selecting a patient for inclusion in treatment or clinical trial with said drug when conditions (i) or (ii) are detected based on the recognition that the genotype combinations (i) and (ii) are associated with a response to said drug, and excluding the patient from treatment or clinical trial with said drug when condition (iii) is detected based on the recognition that the genotype combination (iii) is associated with inadequate response to treatment with said drug.

The method for selecting a human subject for a clinical trial for testing a drug, may alternatively comprises the steps of: (a) assaying a biological sample from a human subject diagnosed with a cartilage disorder for at least the following two single nucleotide polymorphisms: (i) SNP1 and (ii) SNP2, (b) determining the genotypes of the SNPs; (c) selecting for the clinical trial the human subject who carries one of the following genotypes in said SNPs: (i) SNP1 genotype G/G, or C/C in the complement of the SEQ ID NO: 6; and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or NA in the complement of the SEQ ID NO: 7; or (iii) a human subject who does not carry SNP1 genotype G/G, or C/C in the complement of the SEQ ID NO: 6 and SNP2 genotype T/T, or NA in the complement of the SEQ ID NO: 7.

The present invention also describes a method of excluding a human subject from a clinical trial testing a drug, the method comprising the steps of: (a) assaying a biological sample from a human subject diagnosed with a cartilage disorder for at least the following two single nucleotide polymorphisms: (i) SNP1 and (ii) SNP2; (b) determining the genotypes of the SNPs; (c) excluding from the clinical trial the human subject who carries the following genotype in said SNPs: SNP1 genotype G/G, or C/C in the complement of the SEQ ID NO: 6 and SNP2 genotype T/T, or NA in the complement of the SEQ ID NO: 7; or excluding from the clinical trial the human subject who does not carry either of the following SNP genotypes: (i) SNP1 genotype G/G, or C/C in the complement of the SEQ ID NO: 6; and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or A/A in the complement of the SEQ ID NO: 7.

Besides the finding that as a function of his/her genotype, the subject could be classified as super-sensitive, sensitive or non-sensitive, it has surprisingly been found that the same genotype is also predictive of adverse events, such as AIRs. Indeed, further investigations and analysis of the SNP polymorphisms demonstrated a relation between the markers rs9005 and rs315952, in combination, with adverse events in the clinic, with MRI data concerning structural benefit and with symptomatic benefit as determined using the WOMAC questionnaire. Not only these SNPs can be used as predictive tool of the patient's response to a treatment with a drug at cartilage volume level, but can also be used as predictive tool of his/her risk to develop adverse events such as AIRs. Thus, the profile: “structural benefit vs. potential adverse effects” of drug therapy would be useful to determine a better risk/benefit ratio, i.e. better outcome with lower risk of side effects in the patients.

This is indeed based on the finding that the super-sensitives have higher WOMAC scores and higher likelihood for having an AIR event, notably for instance when a drug, such as and FGF-18 compound, is used at a dose of 100 mcg, compared to patients treated with the placebo. Similarly, the non-sensitives also have high WOMAC scores, at any dose, compared to patients treated with the placebo. It has also been shown that contrary to the results a dose of 100 mcg, super-sensitives treated with said drug (such as FGF-18 compound) at a lower dose, for instance 30 mcg, have lower WOMAC scores (i.e. better WOMAC improvement) and lower likelihood of having an AIR event. In view of these results, it can be useful to select the patients based on their likelihood to respond/not respond to a drug treatment in combination with their risk level to present adverse events: the non-sensitives could be excluded from a treatment that is likely not working for them (see above method of selection), and the super-sensitives may be subjects to an alternative treatment regimen.

The present invention is thus also directed to a method for selecting patients having a cartilage disorder for an alternative therapeutic regimen with a drug, based on their likelihood of being super sensitives to said drug treatment, comprising identifying the patient's nucleic acid at both of the polymorphic loci selected from the group consisting of IL-1RN rs9005 and IL-1RN rs315952, wherein the patient's genotype with respect to said loci is predictive about the subject's risk for being super sensitive to a treatment with said drug and allows the selection of said patient for an alternative therapeutic regimen that would be suitable to said patient, in which alternative therapeutic regimen the dose of the drug that is to be administered is reduced compared to the dose of the drug to be administered to a patient who is predicted to be sensitive but not super-sensitive to the said drug treatment.

Also described herein is a method for selecting a patient having a cartilage disorder for a modified treatment regimen with a drug based on the likelihood of said patient of having Acute Inflammatory Reaction (AIR) events when treated with said compound, the method comprising the steps of (a) detecting from a nucleic acid sample obtained from the patient the genotype of (i) SNP1 and (ii) SNP2; and (b) selecting a modified treatment regimen for a patient when a combination of SNP1 genotype NG or AA, or T/C or T/T/ in the complement of the SEQ ID NO: 6; and SNP2 genotype T/T, or NA in the complement of the SEQ ID NO: 7 is detected.

Accordingly, patients having the genotype IL-1 RN rs9005 NG or A/A and IL-1 RN rs315952 T/C or C/C, who are predicted being super-sensitives, are preferably selected for an alternative therapeutic regimen in which one the dose of the drug to be administered is reduced.

Also described herein is a method for selecting patients having a cartilage disorder for an alternative therapeutic regimen with a drug, based on their likelihood of having AIR events when treated with said drug, comprising determining, from a nucleic acid sample, the genotype at both loci IL-1 RN rs9005 and IL-1 RN rs315952, wherein the patient's genotype with respect to said loci is predictive about the subject's risk for developing AIR events in response to treatment with said drug, and allows the selection of said patient for an alternative therapeutic regimen that would be suitable to said patient, in which alternative therapeutic regimen the dose of drug that is to be administered is reduced compared to the dose of drug to be administered to a patient who (1) is predicted to be sensitive and (2) does not present a risk for developing AIR events.

Accordingly, patients having the genotype NG or A/A at IL-1 RN rs9005 and T/C or C/C at IL-1 RN rs315952, who are predicted being super-sensitives, are preferably selected for an alternative therapeutic regimen in which one the dose of drug to be administered is reduced, compared to the normal therapeutic regimen, i.e. the one for a patient who is predicted to be sensitive to drug treatment but who does not present a risk for developing AIR events.

The present invention is also directed to an assay for selecting a treatment regimen for a human subject with a cartilage disorder, the assay comprising: (a) subjecting a test sample from the human subject, who is diagnosed as having cartilage disorder, to at least one genotyping assay that determines the genotypes of at least two loci, wherein said at least two loci are: (i) SNP1 and (ii) SNP2; (b) detecting from the genotypes of said at least two loci the presence of a genotype combination selected from: (i) SNP1 genotype G/G, or C/C in the complement of the SEQ ID NO: 6; and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or A/A in the complement of the SEQ ID NO: 7; or (iii) SNP1 genotype G/G, or C/C in the complement of the SEQ ID NO: 6 and SNP2 genotype T/T, or A/A in the complement of the SEQ ID NO: 7; and (c) selecting, and optionally administering, a treatment regimen comprising an effective amount of a drug when condition (i) or (ii) is detected based on the recognition that the genotype combinations (i) and (ii) are associated with a response to said drug, and excluding the treatment regimen comprising said drug when condition (iii) is detected based on the recognition that the genotype combination (iii) is associated with inadequate response to treatment with said drug.

Also described is a method for treating a human subject with cartilage disorder, comprising administering a composition comprising an effective amount of a drug to a human subject, who is diagnosed to have cartilage disorder, and who is further determined to carry the combination of the single nucleotide polymorphisms (SNPs) selected from: (i) SNP1 genotype G/G, or C/C in the complement of the SEQ ID NO: 6, wherein SNP1 is position X of SEQ ID NO: 6 identified by rs9007, wherein the SEQ ID NO. 6 is a portion of genomic nucleic acid sequence of interleukin 1 receptor antagonist (IL-1 RN); and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or A/A in the complement of the SEQ ID NO: 7, wherein SNP2 is position X of SEQ ID NO. 7 identified by rs317972, wherein the SEQ ID NO. 7 is a portion of genomic nucleic acid sequence of interleukin 1 receptor antagonist (IL-1 RN).

Further discloses is a method for treating a human subject with a cartilage disorder, comprising (a) assaying a biological sample of a subject, who is diagnosed as having the cartilage disorder for at least the following two SNP loci: (i) SNP1, and (ii) SNP2; and (b) administering a treatment regimen comprising a composition comprising an effective amount of an FGF-18 compound to the subject if one of the following conditions is detected: (i) SNP1 genotype G/G, or C/C in the complement of the SEQ ID NO: 6; and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or A/A in the complement of the SEQ ID NO: 7.

Alternatively, the method for treating a human subject with a cartilage disorder, comprises the steps of: (a) assaying a biological sample of a subject, who is diagnosed as having the cartilage disorder for at least the following two SNP loci: (i) SNP1, and (ii) SNP2 and (b) administering a treatment regimen comprising a composition comprising an effective amount of an FGF-18 compound to the subject if SNP1 genotype G/G, or C/C in the complement of the SEQ ID NO: 6 and SNP2 genotype T/T, or A/A in the complement of the SEQ ID NO: 7 is not detected.

In yet an other alternative, the method for selecting in a subject having a cartilage disorder, wherein said a cartilage disorder is susceptible to treatment with a drug, comprises: (a) obtaining a biological sample from the subject with a cartilage disorder with the objective to determine whether the cartilage disorder in the subject is susceptible to treatment with said drug; (b) contacting the biological sample with at least two oligonucleotides capable of interrogating whether or not the biological sample comprises the combination of the single nucleotide polymorphisms (SNPs) selected from (i) SNP1 genotype G/G, or C/C in the complement of the SEQ ID NO: 6, and SNP2 genotype T/C or CC, or NG or GG in the complement of the SEQ ID NO: 7; or (ii) SNP1 genotype NG or AA, or T/C or T/T/ in the complement of the SEQ ID NO: 6; and SNP2 genotype T/T, or A/A in the complement of the SEQ ID NO: 7; (c) identifying the cartilage disorder in the subject as susceptible for treatment with said drug when either the combination of (i) or (ii) is detected in the biological sample and identifying the cartilage disorder in the subject as poorly or non-responsive to treatment with said drug when neither (i) nor (ii) is detected in the biological sample.

Also described herein is a method for selecting a treatment regimen for a subject with a cartilage disorder, comprising: (a) obtaining a test sample from the human subject diagnosed as having depression; (b) subjecting the test sample to at least one analysis to determine parameters of at least two single nucleotide polymorphisms (SNPs), wherein the at least two SNPs comprise the following: (i) SNP1, and (ii) SNP2, (c) detecting using the SNPs, the presence of at least one condition of the following or a combination thereof: i. SNP1 genotype G/G, or C/C in the complement of the SEQ ID NO: 6; and SNP2 genotype T/C or CC, or NG or GG in the complement of the SEQ ID NO: 7; or ii. SNP1 genotype NG or AA, or T/C or T/T/ in the complement of the SEQ ID NO: 6; and SNP2 genotype T/T, or A/A in the complement of the SEQ ID NO: 7; or iii. SNP1 genotype G/G, or C/C in the complement of the SEQ ID NO: 6 and SNP2 genotype T/T, or NA in the complement of the SEQ ID NO: 7 (d) providing a result output setting forth whether at least one of said condition is detected from the test sample and when condition (i) or (ii) is detected, then selecting and optionally administering a treatment regimen comprising a drug to the human subject, and when condition (iii) is detected, then not selecting or administering a treatment regimen comprising said drug to the human subject

In the above mentioned methods, and assay, the patients having the genotype NG or NA at IL-1RN rs9007 (SNP1) and T/C or C/C at IL-1 RN rs317972 (SNP2), who are predicted being super-sensitives, are preferably selected for an alternative therapeutic regimen in which one the dose of the drug to be administered is reduced, compared to the normal therapeutic regimen, i.e. the one for a patient who is predicted to be sensitive to the drug treatment but who does not present a risk for developing AIR events.

In another embodiments of the invention, also provided are systems (and computer readable media for causing computer systems) for obtaining data. Said data can be used notably for assessing suitability of a treatment with a drug in a subject, for assessing the subject's risk of developing AIR when treated with said drug, or monitoring treatment efficacy of a subject with said drug. Said systems can be used during clinical trials, notably when a treatment with a drug for treating a cartilage disorder has to be envisaged or when a treatment with said compound is already ongoing.

Therefore, in an embodiment of the present invention is included a computer system for obtaining data from at least one test sample obtained from at least one subject with a cartilage disorder, the system comprising: (a) at least one determination module configured to receive said at least one test sample and perform at least one analysis on said at least one test sample to determine the presence or absence of the following conditions: (i) SNP1 genotype G/G, or C/C in the complement of the SEQ ID NO: 6, and SNP2 genotype T/C or CC, or A/G or GG in the complement of the SEQ ID NO: 7 or (ii) SNP1 genotype A/G or AA, or T/C or T/T/ in the complement of the SEQ ID NO: 6; and SNP2 genotype T/T, or NA in the complement of the SEQ ID NO: 7; or (iii) SNP1 genotype G/G, or C/C in the complement of the SEQ ID NO: 6, and SNP2 genotype T/T, or NA in the complement of the SEQ ID NO: 7; (b) at least one storage device configured to store data output from said determination module; and (c) at least one display module for displaying a content based in part on the data output from said determination module, wherein the content comprises a signal indicative of the presence of at least one of these conditions, and optionally the absence of any one of these conditions.

Also described is a computer system for obtaining data from at least one test sample obtained from at least one subject, the system comprising: (a) a determination module configured to receive said at least one test sample and perform at least one genotyping analysis on said at least one test sample to determine the genotypes of at least two loci, wherein said at least two loci comprise: (i) SNP1, and (ii) SNP2, (b) a storage device configured to store output data from said determination module; (c) a computing module comprising specifically-programmed instructions to determine from the output data the presence of any of the combinations of polymorphisms selected from the following: i. SNP1 genotype G/G, or C/C in the complement of the SEQ ID NO: 6; and SNP2 genotype T/C or CC, or A/G or GG in the complement of the SEQ ID NO: 7; or ii. SNP1 genotype NG or AA, or T/C or T/T/ in the complement of the SEQ ID NO: 6; and SNP2 genotype T/T, or A/A in the complement of the SEQ ID NO: 7; or iii. SNP1 genotype G/G, or C/C in the complement of the SEQ ID NO: 6 and SNP2 genotype T/T, or A/A in the complement of the SEQ ID NO: 7; and (d) a display module for displaying a content based in part on the data output from said computing module, wherein the content comprises a signal indicative of the presence of the combination (i), (ii), or (iii) of the SNPs, and optionally the absence of any one or more or the combinations (i), (ii), and (iii) of the SNPs.

The computer readable medium can have computer readable instructions recorded thereon to define software modules for implementing a method on a computer. In such a case, said computer readable storage medium may comprise: (a) instructions for comparing the data stored on a storage device with reference data to provide a comparison result, wherein the comparison identifies the presence or absence of at least one of the following conditions: (i) SNP1 genotype G/G, or C/C in the complement of the SEQ ID NO: 6, and SNP2 genotype T/C or CC, or NG or GG in the complement of the SEQ ID NO: 7, or (ii) SNP1 genotype NG or AA, or T/C or T/T/ in the complement of the SEQ ID NO: 6; and SNP2 genotype T/T, or NA in the complement of the SEQ ID NO: 7; or (iii) SNP1 genotype G/G, or C/C in the complement of the SEQ ID NO: 6, and SNP2 genotype T/T, or A/A in the complement of the SEQ ID NO: 7; and (b) instructions for displaying a content based in part on the data output from said determination module, wherein the content comprises a signal indicative of the presence of at least one of the conditions, and optionally the absence of one or more of the conditions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein.

The information determined in the determination module can be read by the storage device. The storage device is adapted or configured for having recorded thereon expression level or protein level information. Such information may be provided in digital form that can be transmitted and read electronically, e.g., via the Internet, on diskette, via USB (universal serial bus) or via any other suitable mode of communication.

It is to be understood that in the context of the present invention as a whole, e.g. in the context of any one of the methods, uses, assays or kits according to the present invention, before determining the genotype at one locus, it is needed to obtain a nucleic acid sample (or a test sample) of one subject, for instance by blood or saliva collecting. Preferably, the nucleic acid sample is a DNA sample.

In the context of the present invention as a whole, e.g. in the context of any one of the methods, uses, computer system or kits according to the present invention, the preferred drug is an anabolic drug, i.e. which have an anabolic effect on cartilage. In particular said anabolic drug is selected from the group consisting of an FGF-18 compound, BMP-2, BMP-7, GDF-5, FGFβ, FGF-8, FGF-9, SOX-9 enhancers, or TGFβ and any variants thereof. More preferably the anabolic drug is a truncated FGF-18 compound, such as sprifermin.

Also in the context of the present invention as a whole, the cartilage disorder is preferably selected from the group consisting of osteoarthritis, cartilage injury, fractures affecting joint cartilage or surgical procedures with impact on joint cartilage (e.g. Microfracture).

An individual afflicted with a cartilage disorder and to be tested and/or treated according to any of the methods and uses described herein is a human subject that is a candidate for treatment with a drug, preferably an anabolic drug, such as sprifermin. In a preferred embodiment, the individual has been diagnosed with cartilage disorder, or exhibits a symptom of cartilage disorder. Said cartilage disorder is preferably selected from the group consisting of, but not limited to, osteoarthritis, cartilage injury, fractures affecting joint cartilage or surgical procedures with impact on joint cartilage (e.g. Microfracture).

It is also to be understand that in the context of the invention as a whole, determination can be performed on the complementary sequence of IL1-RN rs9005 and IL1-RN rs315952. It thus follows that according to the present invention as a whole, e.g. in the context of any one of the methods, uses, assays, computer system or kits according to the present invention, the presence of the genotype C/C on the complementary sequence to IL-1 RN rs9005 and NA on the complementary sequence of IL-1 RN rs315952 is predictive of no response or low response (i.e. non-sensitivity) to treatment with an FGF-18 compound. On the contrary, the presence of the genotype T/C or T/T on the complementary sequence at IL-1 RN rs9005 and NG or G/G on the complementary sequence of IL-1 RN rs315952 is predictive of high response (high-sensitivity) to treatment with an FGF-18 compound. Said genotype will also be a marker of likelihood for a patient of developing AIRs events when treated with said FGF-18 compound. The other genotypes at these loci are predictive of intermediate sensitivity (i.e. C/C in the complement of IL-1 RN rs9005 and NG or G/G in the complement of IL-1 RN rs315952 or T/C or T/T in the complement of IL-1 RN rs9005 and A/A in the complement of IL-1 RN 315952).

In a further embodiment, the present invention encompasses a kit comprising means for performing the methods described above and instructions for use. In particular, the kit comprises at least a couple of specific primers or probes for detecting the presence or absence of the alleles. Preferably, it comprises two couples of specific primers or probes for genotyping the alleles at loci IL-1RN rs9005 and IL-1RN rs315952.

The kit may comprise an oligonucleotide array affixed with a plurality of oligonucleotide probes that interrogate no more than 20 single nucleotide polymorphisms (SNPs), said SNPs comprising: (i) SNP1 genotype G/G, or C/C in the complement of the SEQ ID NO: 6, and SNP2 genotype T/C or CC, or NG or GG in the complement of the SEQ ID NO: 7; or (ii) SNP1 genotype NG or AA, or T/C or T/T/ in the complement of the SEQ ID NO: 6 and SNP2 genotype T/T, or NA in the complement of the SEQ ID NO: 7; or (iii) SNP1 genotype G/G, or C/C in the complement of the SEQ ID NO: 6, and SNP2 genotype T/T, or NA in the complement of the SEQ ID NO: 7; an optional container containing a detectable label to be conjugated to a nucleotide molecule derived from a test sample of a subject diagnosed as having a cartilage disorder; and at least one reagent. Alternatively, the oligonucleotide array affixed with a plurality of oligonucleotide probes interrogates no more than 17 single nucleotide polymorphisms (SNPs), no more than 10 single nucleotide polymorphisms (SNPs) or no more than 7 single nucleotide polymorphisms (SNPs).

Also described in the context of this invention is a kit comprising: a plurality of oligonucleotide primers or sets of primers that each bind to interrogate no more than one specific allele of no more than 20 single nucleotide polymorphisms (SNPs), wherein each subset of oligonucleotide primers that bind to a specific allele of a SNP is labeled with a distinct reporter, and wherein said SNPs comprise the following SNPs: i. SNP1 genotype G/G, or C/C in the complement of the SEQ ID NO: 6, and SNP2 genotype T/C or CC, or NG or GG in the complement of the SEQ ID NO: 7; or ii. SNP1 genotype NG or AA, or T/C or T/T/ in the complement of the SEQ ID NO: 6 and SNP2 genotype T/T, or NA in the complement of the SEQ ID NO: 7; or iii. SNP1 genotype G/G, or C/C in the complement of the SEQ ID NO: 6, and SNP2 genotype T/T, or NA in the complement of the SEQ ID NO: 7; and at least one reagent.

Alternatively, the plurality of oligonucleotide primers or sets of primers that each bind to interrogate no more than one specific allele of no more than 17 single nucleotide polymorphisms (SNPs), or no more than one specific allele of no more than 10 single nucleotide polymorphisms (SNPs) or no more than one specific allele of no more than 7 single nucleotide polymorphisms (SNPs).

In a further embodiment, the present invention discloses a kit for selecting a treatment regimen for a subject with a cartilage disorder, comprising at least one reagent for determining in a test sample of a human subject diagnosed as having the cartilage disorder, the presence or absence of the following SNPs: i. SNP1 genotype G/G, or C/C in the complement of the SEQ ID NO: 6, and SNP2 genotype T/C or CC, or A/G or GG in the complement of the SEQ ID NO: 7; or ii. SNP1 genotype NG or AA, or T/C or T/T/ in the complement of the SEQ ID NO: 6 and SNP2 genotype T/T, or NA in the complement of the SEQ ID NO: 7; or iii. SNP1 genotype G/G, or C/C in the complement of the SEQ ID NO: 6 and SNP2 genotype T/T, or NA in the complement of the SEQ ID NO: 7.

In some embodiments, the oligonucleotides in the kit are either allele-specific probes or allele-specific primers. In other embodiments, the kit comprises primer-extension oligonucleotides. In still further embodiments, the set of oligonucleotides is a combination of allele-specific probes, allele-specific primers, or primer-extension oligonucleotides.

The composition and length of each oligonucleotide in a kit of the invention will depend on the nature of the genomic region containing the genetic marker of the invention as well as the type of assay to be performed with the oligonucleotide and is readily determined by the skilled artisan. For example, the polynucleotide to be used in the assay may constitute an amplification product, and thus the required specificity of the oligonucleotide is with respect to hybridization to the target region in the amplification product rather than in genomic DNA isolated from the individual.

In preferred embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting polymorphisms, departures from complete complementarity are contemplated where such departures do not prevent the molecule from specifically hybridizing to the target region as defined above. For example, an oligonucleotide primer may have a non-complementary fragment at its 5′ end, with the remainder of the primer being completely complementary to the target region. Alternatively, non-complementary nucleotides may be interspersed into the probe or primer as long as the resulting probe or primer is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. As the target sequences are generally present in excess, at Tₘ, 50% of the probes are occupied at equilibrium. Typically, stringent conditions include a salt concentration of at least about 0.01 to 1.0 M Na ion concentration (or other salts) at pH 7.0 to 8.3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide [peptide nucleic acid (PNA)] and the like. The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may be constitute components of an approved diagnostic device.

In other preferred embodiments, the kit includes an instruction manual that describes the various ways the kit may be used to detect the presence or absence of a genetic marker of the invention. In a preferred embodiment, the set of oligonucleotides in the kit are allele-specific oligonucleotides. As used herein, the term allele-specific oligonucleotide (ASO) means an oligonucleotide that is able, under sufficiently stringent conditions, to hybridize specifically to one allele of a genetic marker, at a target region containing the genetic marker while not hybridizing to the same region containing a different allele. As understood by the skilled artisan, allele-specificity will depend upon a variety of readily optimized stringency conditions, including salt and formamide concentrations, as well as temperatures for both the hybridization and washing steps.

Typically, an ASO will be perfectly complementary to one allele while containing a single mismatch for another allele. In ASO probes, the single mismatch is preferably within a central position of the oligonucleotide probe as it aligns with the genetic marker in the target region (e.g., approximately the 7th or 8th position in a 15mer, the 8th or 9th position in a 16mer, and the 10th or 11th position in a 20mer). The single mismatch in ASO primers is located at the 3′ terminal nucleotide, or preferably at the 3′ penultimate nucleotide. ASO probes and primers hybridizing to either the coding or non-coding strand are contemplated by the invention.

In other preferred embodiments, the kit comprises a pair of allele-specific oligonucleotides for a genetic marker of the invention to be assayed, with one member of the pair being specific for one allele and the other member being specific for another allele. In such embodiments, the oligonucleotides in the pair may have different lengths or have different detectable labels to allow the user of the kit to determine which allele-specific oligonucleotide has specifically hybridized to the target region, and thus determine which allele is present in the individual at the assayed marker locus.

In still other preferred embodiments, the oligonucleotides in the kit are primer-extension oligonucleotides. Termination mixes for polymerase-mediated extension from any of these oligonucleotides are chosen to terminate extension of the oligonucleotide at the genetic marker of interest, or one base thereafter, depending on the alternative nucleotides present at the marker locus.

The methods and kits according to the present invention are useful in clinical diagnostic applications. However, as used herein, the term “diagnostic” is not limited to clinical or medical uses, and the diagnostic methods and kits of the invention claimed herein are also useful in any research application, and during clinical trials, for which it is desirable to test a subject for the presence or absence of any genetic marker described herein.

In the context of the invention, the presence or absence of a particular allele or pair of alleles at the locus of a genetic marker of the invention in an individual may be detected by any technique known per se to the skilled artisan, including sequencing, pyrosequencing, selective hybridization, selective amplification and/or mass spectrometry including matrix-assisted laser desorption/ionization time-of-flight mass spectrometry (MALDI-TOF MS). In a particular embodiment, the alteration is detected by selective nucleic acid amplification using one or several specific primers. The alteration is detected by selective hybridization using one or several specific probes.

Further techniques include gel electrophoresis-based genotyping methods such as PCR coupled with restriction fragment length polymorphism (RFLP) analysis, multiplex PCR, oligonucleotide ligation assay, and minisequencing; fluorescent dye-based genotyping technologies such as oligonucleotide ligation assay, pyrosequencing, single-base extension with fluorescence detection, homogeneous solution hybridization such as TaqMan, and molecular beacon genotyping; sequencing-based technologies such as Sanger sequencing and next-generation sequencing platforms; rolling circle amplification and Invader assays as well as DNA chip-based microarray and mass spectrometry genotyping technologies. Protein expression analysis methods are known in the art and include 2-dimensional gel-electrophoresis, mass spectrometry and antibody microarrays. Sequencing can be carried out using techniques well known in the art, e.g. using automatic sequencers. The sequencing may be performed on the complete gene or, more preferably, on specific domains thereof, typically those known or suspected to carry deleterious mutations or other alterations.

Amplification may be performed according to various techniques known in the art, such as by polymerase chain reaction (PCR), ligase chain reaction (LCR) and strand displacement amplification (SDA). These techniques can be performed using commercially available reagents and protocols. A preferred technique is allele-specific PCR.

Other embodiments of the invention within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### DESCRIPTION OF THE FIGURES

General notes: in the figures, 1) the terms TT, CC, GG or AA are to be understood as being T/T, C/C, G/G or NA, and 2) the term CTA is to be understood as C-T-A.

FIG. 1: Organization of the IL1R1-IL1A-IL1B-IL1 RN gene cluster. Both rs315952 and rs9005 are located in the last IL1RN exon. Although there is only 1107 bp between them, these SNPs are not inherited together (i.e. not in Linkage Disequilibrium). IL1RN-rs9005 is within the 3′ UTR region and overlaps both a transcription factor (ChIP-seq sequence: FOSL2) and a DNAse cluster (regulatory regions and promoter tend to be DNAse sensitive). IL1 RN-rs315952 is a coding silent SNP (i.e. does not lead to an amino acid change).

FIG. 2: Stratification of the patients as a function of presence or absence (from at least one copy) of the C-T-A haplotype. The Y axis shows change at Week 52 in total cartilage volume (unit: mm³). Each point corresponds to a subject, circle indicates a subject without AIR while cross indicates a subject with AIRs. Indicated p-value was obtained from a non-parametric univariate test (ranksum test).

FIG. 3: Stratification of the patients as a function of presence or absence (from at least one copy) of the C-T-A haplotype. The Y axis shows change at Week 52 in WOMAC total score. Each point corresponds to a subject, circle indicates a subject without AIR event while cross indicates a subject with AIRs. Indicated p-value was obtained from a non-parametric univariate test (ranksum test).

FIG. 4: Change in total cartilage volume (mm³) at Week 52 stratified by dose regimen and stratified by their genotype at both rs315952 and rs9005. Each point corresponds to a subject, circle indicates a subject without AIR while cross indicates a subject with AIRs. MRI data from the MAD010 cohort showed aberrant variability and were not included in any analyses.

FIG. 5: Change in WOMAC total score at Week 52 stratified by dose regimen and stratified by their genotype at both rs315952 and rs9005. Each point corresponds to a subject, circle indicates a subject without AIR while cross indicates a subject with AIRs.

FIG. 6: Stratification of the patients as a function of presence or absence of the ‘rs9005 G/G rs315952 T/T’ genotype. The Y axis shows absolute WOMAC total score at baseline. Each point corresponds to a subject, circle indicates a subject with Kellgren-Lawrence grade equals to 2 while cross indicates a subject with Kellgren-Lawrence grade equals to 3. Indicated p-value was obtained from a non-parametric univariate test (ranksum test).

FIG. 7: Stratification of the patients as a function of presence or absence of the ‘rs9005 A carriers rs315952 C carriers’ genotype. The Y axis shows absolute WOMAC total score at baseline. Each point corresponds to a subject, circle indicates a subject with Kellgren-Lawrence grade equals to 2 while cross indicates a subject with Kellgren-Lawrence grade equals to 3. Indicated p-value was obtained from a non-parametric univariate test (ranksum test).

FIG. 8: Stratification of the patients as a function of presence or absence of the ‘rs9005 G/G rs315952 T/T’ genotype. The Y axis shows absolute total cartilage volume (mm³) at baseline. Each point corresponds to a subject, circle indicates a subject with Kellgren-Lawrence grade equals to 2 while cross indicates a subject with Kellgren-Lawrence grade equals to 3. Indicated p-value was obtained from a non-parametric univariate test (ranksum test).

FIG. 9: Stratification of the patients as a function of presence or absence of the ‘rs9005 A carriers rs315952 C carriers’ genotype. The Y axis shows absolute total cartilage volume (mm³) at baseline. Each point corresponds to a subject, circle indicates a subject with Kellgren-Lawrence grade equals to 2 while cross indicates a subject with Kellgren-Lawrence grade equals to 3. Indicated p-value was obtained from a non-parametric univariate test (ranksum test).

FIG. 10: Change from baseline in WOMAC total score for all subjects irrespectively of their genotypes. Lines correspond to the mean change from baseline and error bars correspond to standard error of mean.

FIG. 11: Change from baseline in WOMAC total score for subjects identified as sensitives or super-sensitives based on their rs9005 and rs315952 genotypes. The ‘treated’ group corresponds to subjects from the MAD100 cohort having the genotype identifying sensitive subjects. Subjects from the MAD030 cohort having the genotype identifying super-sensitive subjects are also included in this ‘treated’ group. The ‘placebo’ group includes placebos subjects with genotype corresponding to either the sensitives or to the super-sensitives. Lines correspond to the mean change from baseline and error bars correspond to standard error of mean.

FIG. 12: Change from baseline in WOMAC total score for subjects having the genotype corresponding to the non-sensitives. Lines correspond to the mean change from baseline and error bars correspond to standard error of mean.

FIG. 13: Change from baseline in total cartilage volume (mm³) for all subjects irrespectively of their genotypes. Lines correspond to the mean change from baseline and error bars correspond to standard error of mean. MRI data from the MAD010 cohort showed aberrant variability and were not included in any analyses.

FIG. 14: Change from baseline in total cartilage volume (mm³) for subjects identified as sensitives or super-sensitives based on their rs9005 and rs315952 genotypes. The ‘treated’ group corresponds to subjects from the MAD100 cohort having the genotype identifying sensitives. Subjects from the MAD030 cohort having the genotype identifying super-sensitives are also included in this ‘treated’ group. The ‘placebo’ group includes placebos subjects with genotype corresponding to either the sensitives or to the super-sensitives. Lines correspond to the mean change from baseline and error bars correspond to standard error of mean. MRI data from the MAD010 cohort did not passed quality control and were not included in any analyses.

FIG. 15: Change from baseline in total cartilage volume (mm³) for subjects having the genotype corresponding to the non-sensitives. Lines correspond to the mean change from baseline and error bars correspond to standard error of mean. MRI data from the MAD010 cohort showed aberrant variability and were not included in any analyses.

FIG. 16(a)-(h): Sets out the full length amino acid and nucleic acid sequences corresponding to the “SEQ ID NOs” referenced in the instant patent application.

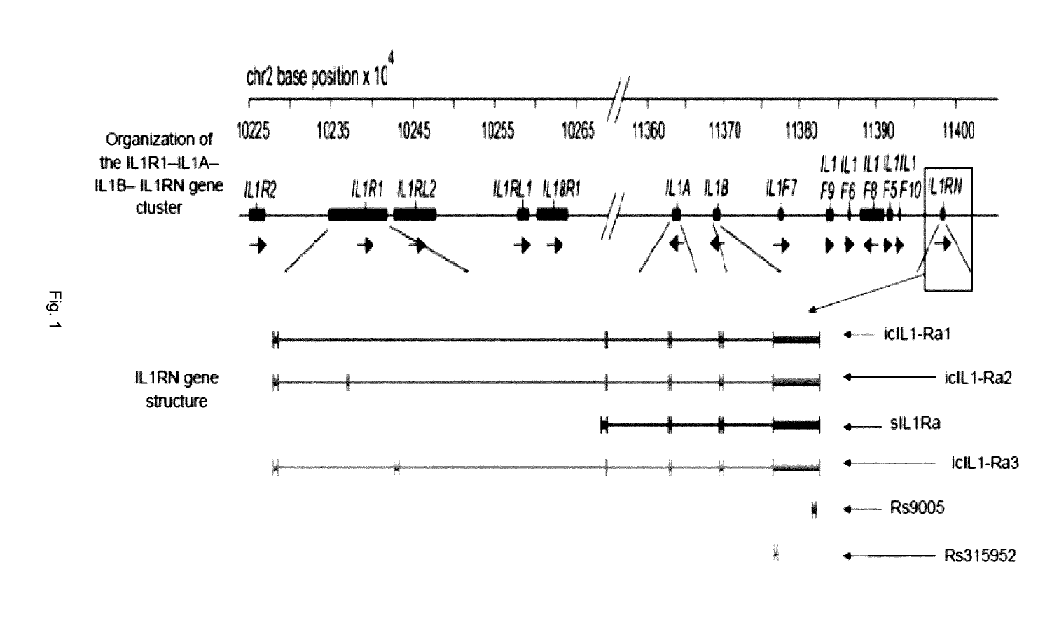

### DESCRIPTION OF THE SEQUENCES

SEQ ID NO. 1: Amino acid sequence of the native human FGF-18.
SEQ ID NO. 2: Amino acid sequence of the recombinant truncated FGF-18 (trFGF-18).
SEQ ID NO. 3: IL1RN gene
SEQ ID NO. 4: IL1RN rs9005 locus
SEQ ID NO. 5: IL1RN rs315952 locus
SEQ ID NO. 6: Specific region from URN rs9005 locus (corresponding to nucleotide 415 to nucleotide 466 of SEQ ID NO. 4), wherein N is A or G
SEQ ID NO. 7: Specific region from URN rs315952 locus (corresponding to nucleotide 415 to nucleotide 466 of SEQ ID NO. 5), wherein N is C or T
SEQ ID NO. 8: rs315952 primer 1
SEQ ID NO. 9: rs315952 primer 2
SEQ ID NO. 10: rs9005 primer 1
SEQ ID NO. 11: rs9005 primer 2

### Examples

### 1. Genotyping Background

The level of cartilage volume growth and the associated risk of adverse events in response to a drug treatment (such as sprifermin treatment) in cartilage disorders, such as osteoarthritis, cartilage injury, fractures affecting joint cartilage or surgical procedures with impact on joint cartilage (e.g. Microfracture), may each be associated with a specific genetic variation in one or several genes. In the present study, the search for associations between genes containing variations and disease or response to treatment was focused on candidate genes that were selected based on the physiological role of the proteins they encode and their potential implication in the cartilage disorders, or in the response to sprifermin treatment. The list of selected candidate SNPs that have been tested is given in Table 1.

Response to sprifermin treatment was measured by change in cartilage volume from baseline 1 year after the beginning of treatment with sprifermin.

It is noted that candidate and whole genome scan SNP markers were not kept for further analysis if any of the following criteria was met:
Rare variant SNP in the PGx ITT population: Minor Allele Frequency (MAF)<10% for both candidate SNP and whole genome scan SNPs.
Questionable genotyping quality, as measured by a high rate (≧5%) of missing data.
Significant deviation from the Hardy-Weinberg equilibrium (Bonferroni adjusted p value less than 5% for candidate SNPs or FDR (i.e. Benjamini-Hochberg adjusted p value) less than 20% for whole genome scan SNPs).
Subjects with gender discrepancy between the clinical database and the predicted gender from whole genome scan SNP data (chromosome X) are excluded.

The candidate genes selected have been previously implicated in cartilage disorder, such as osteoarthritis. The purpose of the study was to investigate whether the level of response, i.e. cartilage volume growth and/or occurrence of adverse events in response to sprifermin treatment in cartilage disorder is correlated with a specific DNA variant or pattern of variants. The existence of such a correlation would indicate that either the gene(s) carrying the identified variant(s) or one or more genes lying in the vicinity of the variants may be (a) susceptibility gene(s).

### 2 Materials and Methods

### 2.1. FGF-18 Compound

The FGF-18 compound used as a treatment in the present examples is sprifermin. It is a truncated form of FGF-18, as defined in the section “definitions”.

### 2.2. Sample Reception and Double Coding

Blood samples were received from patients participating in study 28980 (A randomized, double blind, placebo-controlled, multicenter, single and multiple ascending dose study of sprifermin, administered intra-articularly in patients with primary osteoarthritis of the knee who are not expected to require knee surgery within one year)

In order to comply with the Pharmacogenomics (PGx) Informed Consent Form (ICF), which covered the DNA analysis, all samples were double-coded by the Biobank (Merck Serono, Geneva) to ensure an additional level of subject anonymity. The Biobank provided the Biomarker Data Management group with the double key coding as a flat file containing both the PGx ID and the Subject ID for each subject. Additional verifications were performed to ensure that no DNA analyses are performed on subjects who did not consent to the PGx study.

### 2.3. DNA Samples Extraction, Amplification, Fragmentation and Labeling

The analysis was performed on DNA extracted from blood. A total of 140 blood samples were received. Out of these 140, 3 samples were destroyed by the genomic laboratory as the patients withdrew their consent during the course of study; resulting in 137 DNAs analyzed corresponding to 137 patients. Thus 137 patients were genotyped and eligible for the association studies.

Genomic DNA was extracted from EDTA blood samples using a Qiagen extraction kit (QIAamp DNA Blood Maxi Kit). After extraction, measures of sample absorbance at wavelengths of 260 nm and 280 nm using a spectrophotometer and electrophoresis on agarose gels were performed to estimate the quality and quantity of genomic DNA samples.

For each plate, genomic DNA samples were digested with Nsp I and Sty I restriction endonucleases, ligated with specific adaptors (Nsp or Sty), processed in parallel until the Polymerase Chain Reactions (PCR). PCR amplified the product of ligation in triplicate for StyI reactions and in quadruplicate for NspI reactions, to product a large efficiency. All the PCR products were pooled, purified, quantified, fragmented and labeled.

PCR amplification step was evaluated using electrophoresis agarose gel. DNA quantification step was measured using spectrophotometer and DNA fragmentation step was evaluated using electrophoresis agarose gel. The average of DNA fragment size should be lower than 180 bp.

### 2.4. DNA Microarray Technology (Whole Genome Scan)

The Affymetrix Genome Wide SNP 6.0 Assays were used to perform the Whole Genome Scan (hypothesis free approach). The Affymetrix technology is based on a DNA chip allowing the genotyping of approximately 906 600 single nucleotide polymorphisms (SNPs) per patient. SNPs are randomly distributed in all the chromosomes and are used as tagging markers of the corresponding genomic area. The details of process and protocol followed the PGX Affymetrix wide-genome SNP 5.0/6.0 technology.

For each sample, the labeled product was hybridized into the Affymetrix Genome Wide SNP 6.0 GeneChip. Two lots of chips were used for both sets.

After hybridization and staining, the Affymetrix Gene Chips were scanned to create image data (DAT) files. After that, AGCC Software aligned automatically a grid on the DAT files and computed the Cell Intensity data (CEL) file. Afterwards the CEL data passed on to Genotyping Console software that generated Probe Analysis (CHP) data.

Analysis quality control was performed using Genotyping Console Software assessing the Dynamic Model QC (DM) call rate analysis of a subset of 3022 SNPs following chip scanning. DM call rates measure the consistency of intensities within each SNP, with four possible genotyping states (Null, AA, AB and BB). It provides an estimate of the overall quality for a data sample prior to performing full clustering analysis. It is based on QC Call Rate.

The QC Call Rate (QC CR) is well correlated with clustering performance and is an effective single-sample metric for deciding what samples should be used in downstream clustering. The fixed threshold for Genome wide SNP6.0 arrays is >=86%. In addition to QC CR, another algorithm has been developed for SNP 6.0 arrays. This new algorithm is the Contrast QC. The contrast QC is a metric that captures the ability of an experiment to resolve SNP signals into three genotype clusters. It measures the separation of allele intensities into three clusters in “contrast space”. Contrast space is a projection of the two-dimensional allele intensity space into an informative single dimension. The default threshold is >=0.4 for each sample. The results of QC are automatically displayed in the Intensity QC Table. Samples, which pass the QC threshold, (call rate>86% and contrast QC>0.4) are noted “bound in”, and those, which did not pass the QC (call rate<86% or contrast QC<0.4) are noted “bound out”. The genomic DNA samples of study passed all QC.

Genome-wide scan were carried to identify SNPs marginally associated with acute inflammatory reactions (AIRs); change in cartilage volume and change in WOMAC total scores. The list of SNPs with marginal association (empirical p-value<5% after n=1000 permutations) can be consulted from Table 1. No SNPs were marginally associated with change in total cartilage volume.

### 2.5. TaqMan SNP Genotyping (Candidate Gene)

TaqMan SNP Genotyping was performed to detect selected markers based on literature information. A total of 19 SNPs distributed onto 8 candidate genes were selected and carried out in two periods (see Tables 2a and 2b). In a TaqMan® SNP Genotyping assay, two locus-specific PCR primers surrounding the SNP are used to amplify a fragment of about 100 bp (see for instance Table 3). Two allele-specific probes are then hybridized to their specific SNP sequence. Each probe was labeled at its 5′ extremity with either a fluorescent reporter dye (FAM), either the VIC reporter dye. Each probe also has a non-fluorescent quencher dye, MGB, at the 3′ end. In each PCR cycle, if the target sequence of the allele-specific probe is amplified, the probe will hybridize to the DNA during the annealing step and extend. When the DNA polymerase comes into contact with this hybridized probe, the reporter dye of the probe is cleaved from the probe leaving the quencher dye behind. In each cycle of the PCR, cleavage of the reporter dyes from one or both of the allele-specific probes causes an exponential increase in the fluorescent intensity. At PCR completion, the total fluorescence of each sample is read on the ABI 9700 (384-well format). If fluorescence is observed from only one probe, the sample is homozygous for this allele. If fluorescence is observed for both allele-specific probes, the sample is heterozygous for both alleles. If the probe does not hybridize, the fluorescence of the dye is “quenched” or reduced by the quencher dye, and thus minimal fluorescence is observed, indicating a failed genotype.

Protocol is detailed in the datasheet of TaqMan® SNP Genotyping.

Period 1: DNA samples were genotyped with 17 TaqMan® SNP assays (see Table 2a).
Period 2: DNA samples were genotyped with 2 further TaqMan® SNP assays (see table 2b).
For each TaqMan® SNP assays, the NTC cluster was specific and all NTCs were undetermined, the three distinct sample clusters were present and genotyping was automatically assigned and the call rate was specified to be above 85 percent.
For each of the 19 TaqMan® SNP assays in the three parts, acceptance criteria were reached.

### 2.6. SNP Filtering

Candidate and whole genome scan SNP markers were not kept for analysis if any of the following criteria was met:
Rare variant SNP in the PGx ITT population: Minor Allele Frequency (MAF)<10% for both candidate SNP and whole genome scan SNPs.
Questionable genotyping quality, as measured by a high rate (≧5%) of missing data.
Significant deviation from the Hardy-Weinberg equilibrium (Bonferroni adjusted p value less than 5% for candidate SNPs or FDR (i.e. Benjamini-Hochberg adjusted p value) less than 20% for whole genome scan SNPs).
Subjects with gender discrepancy between the clinical database and the predicted gender from whole genome scan SNP data (chromosome X) are excluded.

### 2.7. Association Tests

For association tests, genotype data were coded as presence/absence of the SNP minor allele (i.e. homozygous for major allele compared to at least one copy of the minor allele).

2.7.1. Association with Acute Inflammatory Reactions (AIRs)

In these analyses, only subjects treated with 100 mcg FGF18 dose were used. For single marker analysis, two approaches were used: Fisher's exact test and a multivariate linear model (i.e. AIR status˜SNP+Kellgren Lawrence grade[2; 3]+Gender [Female; Male]+Age [<65; ≧65]+BMI [<30, ≧30]. In this model, significance of each term in the model was assessed with a type III anova).

2.7.2. Association with WOMAC Total Scores and Total Cartilage Volume

Association between change from baseline at week 52 (termination date), both for WOMAC total scores and total cartilage volume, was assessed using the following linear model:

Rank(change in endpoint)˜Arm [Placebos, Treated subjects for .e.g. with FGF-18 100 mcg dose]+genotype group+Kellgren Lawrence grade[2; 3]+Gender [Female; Male]+Age [<65; ≧65]+BMI [<30, ≧30].

Significance of each term in the model was assessed with a type III anova and significance threshold was set at alpha=5%.

### 2.7.3. Association Between a Given Genotype Group and Kellgren-Lawrence Grade

To test whether a given genotype group (for e.g. subjects with the ‘IL-1RN rs9005 G/G and IL-1 RN rs315259 T/T’ genotype) had a significant enrichment or paucity in subjects with severe osteoarthritis (i.e. Kellgren-Lawrence grade 3) independence tests were performed using a Fisher's exact test and from the following contingency table:

| | Grade 3 | Grade 2 |
|---|---|---|
| # of subjects from a given | | |
| genotype group | | |
| # of subjects from the | | |
| remaining genotype groups | | |

All available subjects from any dose regimen (including placebos) were included in this analysis. P-values were computed using a two-sided test and significance was set at alpha=5%. Odds ratio and their 95% confidence intervals were also computed.

### 2.8. Haplotype Analyses

Genotype data from SNPs rs419598, rs315952, rs9005 were phased (using the MACH software, version 1.0.18.c, Li Y et al., 2010) to infer presence or absence of the C-T-A haplotype in subjects. The following MACH parameters were used: “--rounds 50 --states 200 --phase”. Association with AIRs was tested using a Fisher's exact test (significance threshold set at alpha=5%).

### 2.9. Combinatorial Analyses Between Candidate SNPs

In initial association analyses (data not shown), the rs9005 SNP was found significantly associated with AIRs. Combinatorial analyses (i.e. epistasis) were performed to test whether IL-1 RN rs9005 in combination of another SNP, from a list of about 120 candidate SNPs, would be a better AIR predictor (see Table 1). Such analysis was performed using a logistic regression with the following model:

AIR status˜rs9005* another SNP+Kellgren Lawrence grade[2; 3]+Gender [Female; Male]+Age [<65; ≧65]+BMI [<30, ≧30].

Significance of each term in the model was assessed with a type III anova. Interaction p-values were adjusted for multiple-testing using the Benjamini-Hochberg procedure (Benjamini and Hochberg, 1995, J. of the Royal Statistical Society Series B(57):289) and significance threshold was set at FDR=5%. Epistasis effects were confirmed using the statistical approach described in (Wirapati et al., 2011).

### 2.10. Performance Metrics at Predicting AIRs

Performance metrics at predicting AIRs were derived from the corresponding contingency table. These metrics included sensitivity, specificity, accuracy, precision, negative predictive value and F1 score (i.e. harmonic mean of precision and recall).

### 3. Results

### 3.1. Predictive Analyses

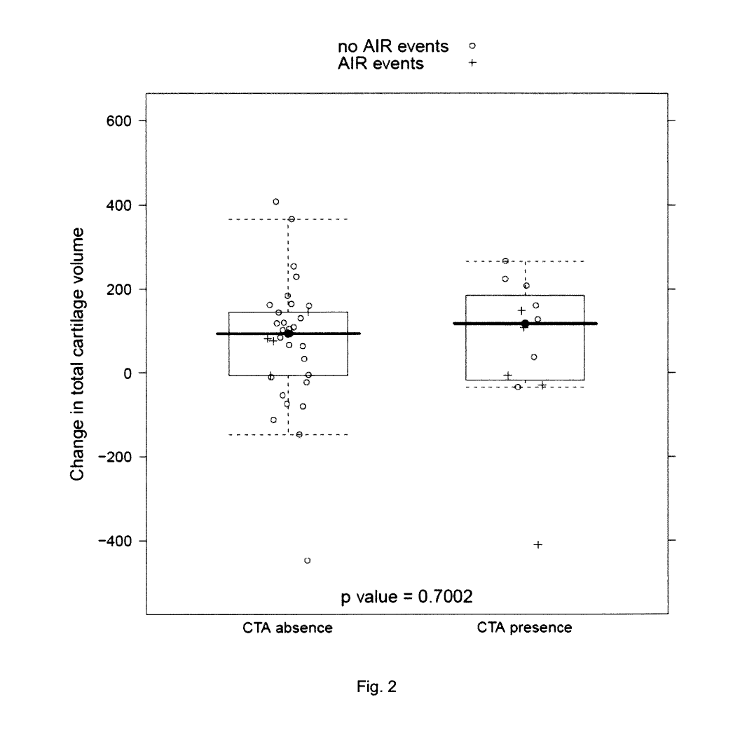

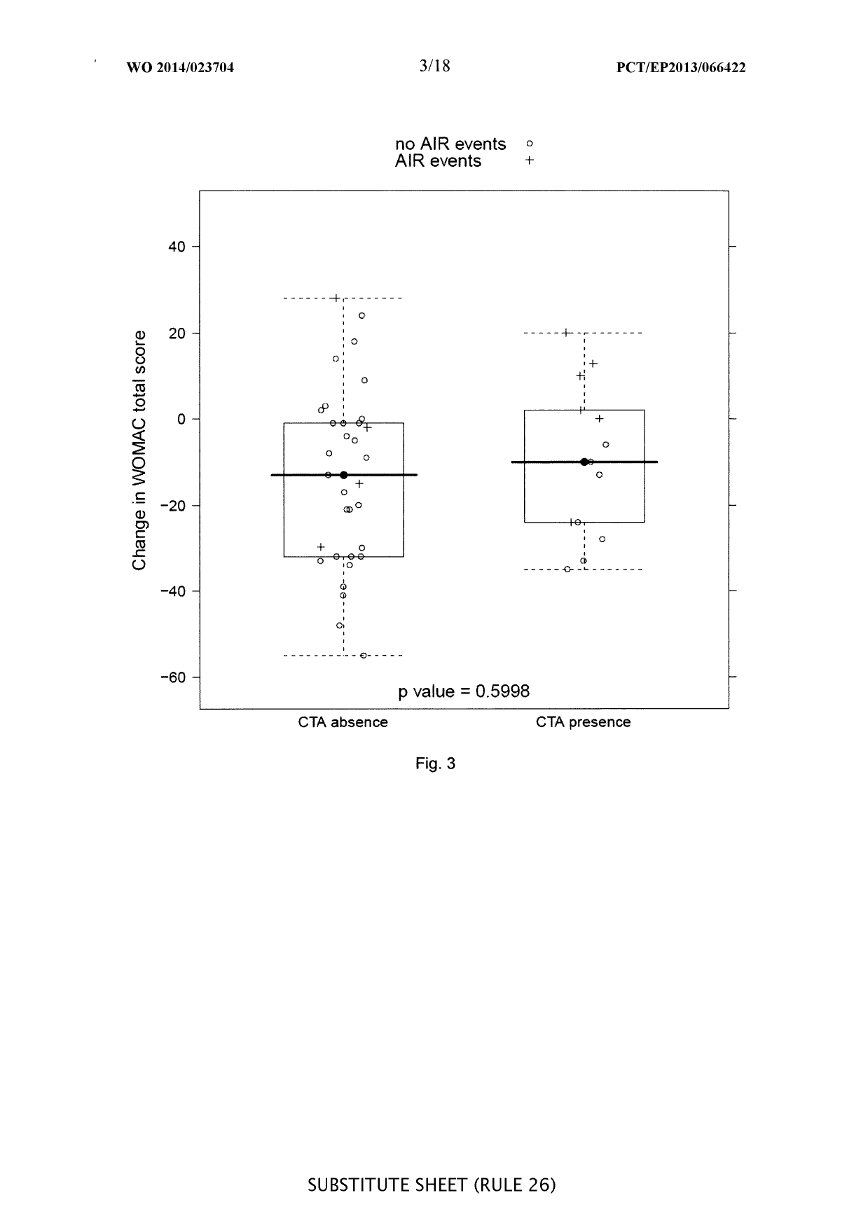

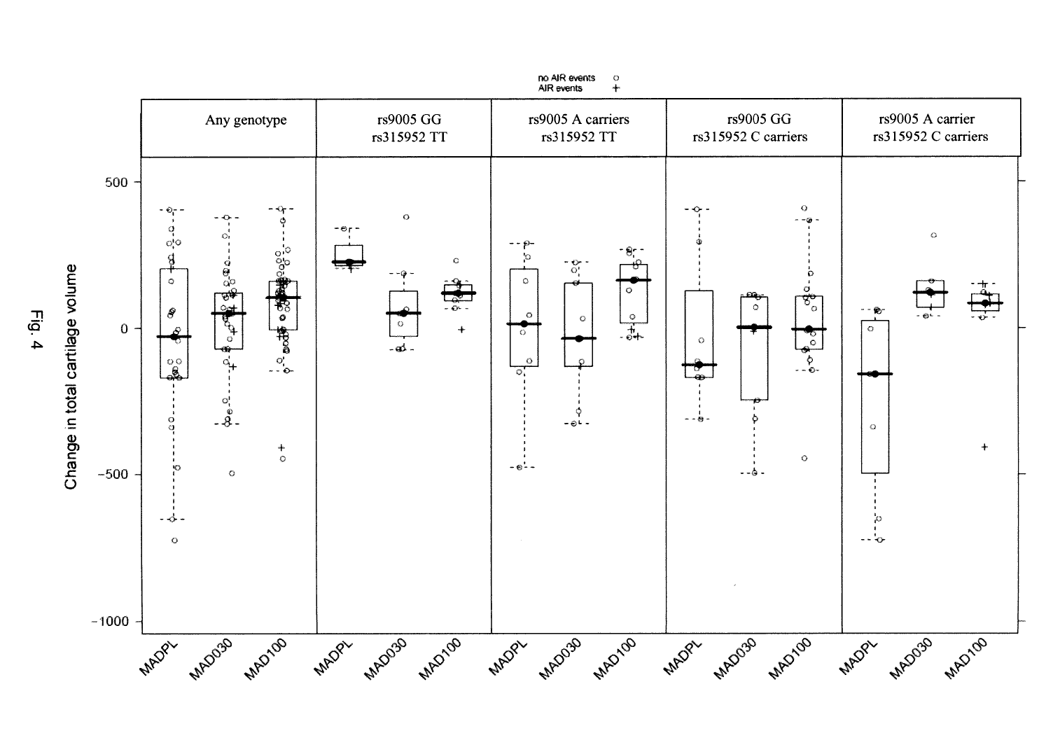

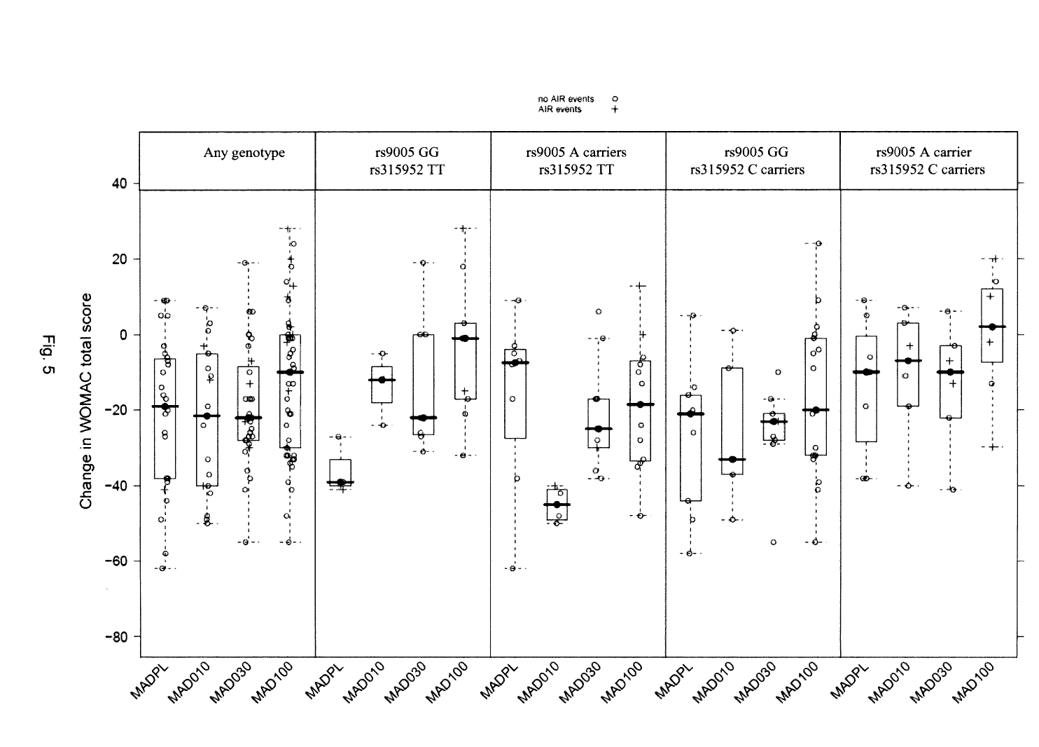

Combinatorial analyses identified only one combination (IL-1 RN rs9005 and IL-1 RN rs315259) as significantly associated with AIRs (FDR from multivariate linear model=0.0187, Fisher's exact test p-value=0.0018, odds ratio=18.82 [2.25-260.03]). Contingency table and prediction performance metrics are shown respectively in Table 5 and Table 6. The combination of rs9005 and rs315259 (Table 6) has a better performance at predicting AIRs, compared to the C-T-A haplotype (Table 8 see also contingency table in Table 7). The combination of IL-1 RN rs9005 and IL-1 RN rs315259 has a very strong specificity (94.44%) and negative predictive value (89.47%), i.e. these biomarkers have a very strong performance at identifying subjects that will not have AIRs. In addition, this combination reveals stratification on total cartilage volume (FIG. 4) and WOMAC total scores (FIG. 5). By contrast, the C-T-A haplotype does not allow such clinical outcome stratification (FIGS. 2 and 3). Indeed, the C-T-A haplotype did not allow stratifying subjects for change in total cartilage volume (FIG. 2) nor change in WOMAC total score (FIG. 3). Thus the C-T-A haplotype was not identified as a good predictor of the response to drug therapy, preferably an anabolic drug such as sprifermin.

### 3.2. Prognostic Analyses

Placebo subjects with the ‘IL-1RN rs9005 G/G and IL-1 RN rs315259 T/T’ genotype were identified as having significantly higher total cartilage volume than treated subjects from the same genotype group. To follow-up on this result, change in WOMAC total score and change in total cartilage volume were modeled in placebo subjects with the following formula:

Rank(change in endpoint)˜genotype group+Kellgren Lawrence grade[2;3]+Gender[Female;Male]+Age[<65;≧65]+BMI[<30,≧30].

No significant difference in WOMAC total score was found between subjects from the four different genotype groups (p-value=0.63, Table 10). However significant differences were found in change in total cartilage volume (p-value=0.02, Table 9). Subjects from the ‘IL-1RN rs9005 G/G and IL-1RN rs315259 T/T’ genotype group have significantly higher total cartilage volume increase compared to subjects from the remaining genotype groups.

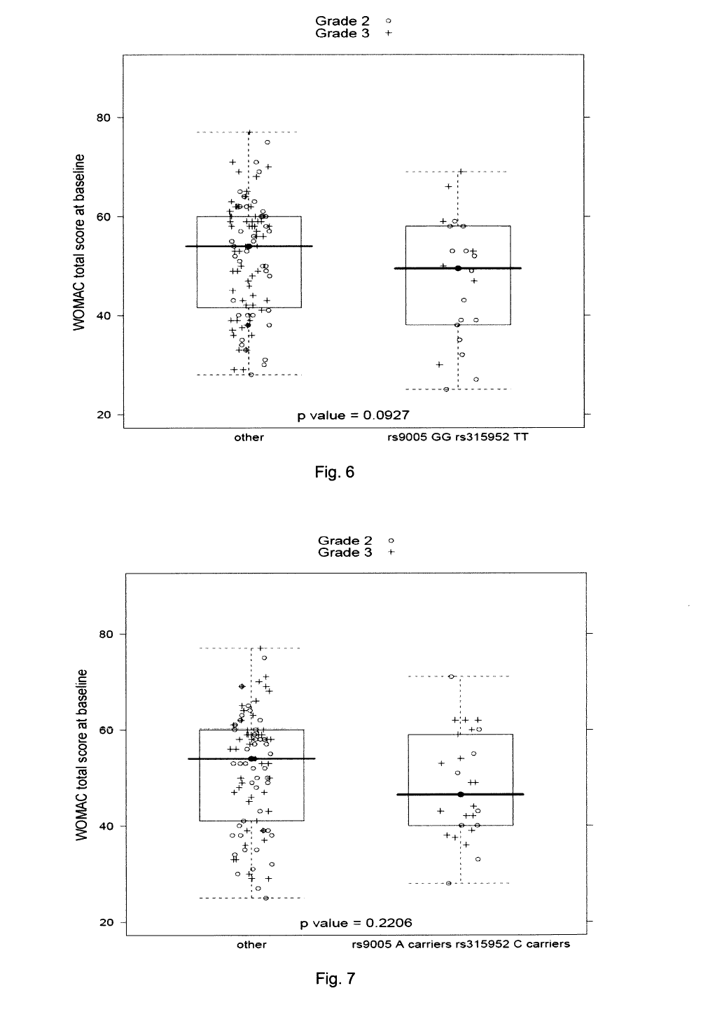

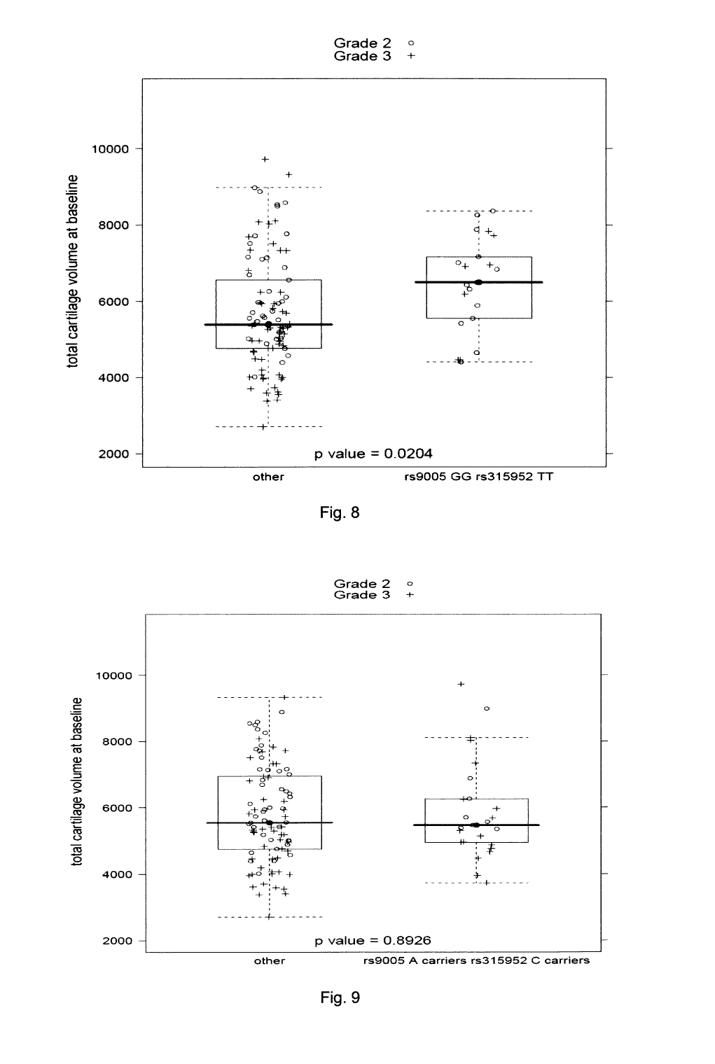

Independence test between the Kellgren-Lawrence grade and subjects from a given genotype group demonstrated that the ‘IL-1RN rs9005 G/G and IL-1 RN rs315259 T/T’ genotype group has a significant paucity in subjects from Kellgren-Lawrence grade 3 (Fisher's exact test p-value=0.0179, Table 11). The corresponding odds ratio is 0.306 (with 95% confidence intervals [0.096, 0.885]). This demonstrates that subjects from the ‘IL-1RN rs9005 G/G and IL-1 RN rs315259 T/T’ genotype group are classified with a less severe osteoarthritis condition than subjects from other genotype groups. Lending support to this result, subjects from the ‘IL-1RN rs9005 G/G and IL-1 RN rs315259 T/T’ genotype group have marginally smaller baseline WOMAC total scores than subjects from other genotype groups (ranksum p-value=0.0927, see FIG. 6). In addition, subjects from the ‘IL-1RN rs9005 GG and IL-1 RN rs315259 TT’ genotype group have significantly higher baseline total cartilage volume than subjects from other genotype groups (ranksum p-value=0.0204, see FIG. 8).

Interestingly, there was no difference in the proportion of subjects with Kellgren-Lawrence grade 3 between the ‘IL-1RN rs9005 A carriers and IL-1RN rs315259 C carriers’ genotype group (aka super-sensitives) and subjects from the remaining genotype groups (Fisher's exact test p-value=0.2736, odds ratio=1.693 [0.637, 4.769], Table 12). Thus the super-sensitive group is not enriched in subjects with severe osteoarthritis condition. This is further enforced with the fact that both baseline WOMAC total scores and baseline total cartilage volume are comparable between super-sensitives subjects and other subjects (see FIGS. 7 and 9).

Analysis with the C-T-A haplotype, did not reveal difference in the proportion of subjects with Kellgren-Lawrence grade 3 and bearing at least one copy of the C-T-A haplotype (Fisher's exact test p-value=1).

### 3.3. Clinical Outcome Using the Proposed Genetic Diagnostic Test

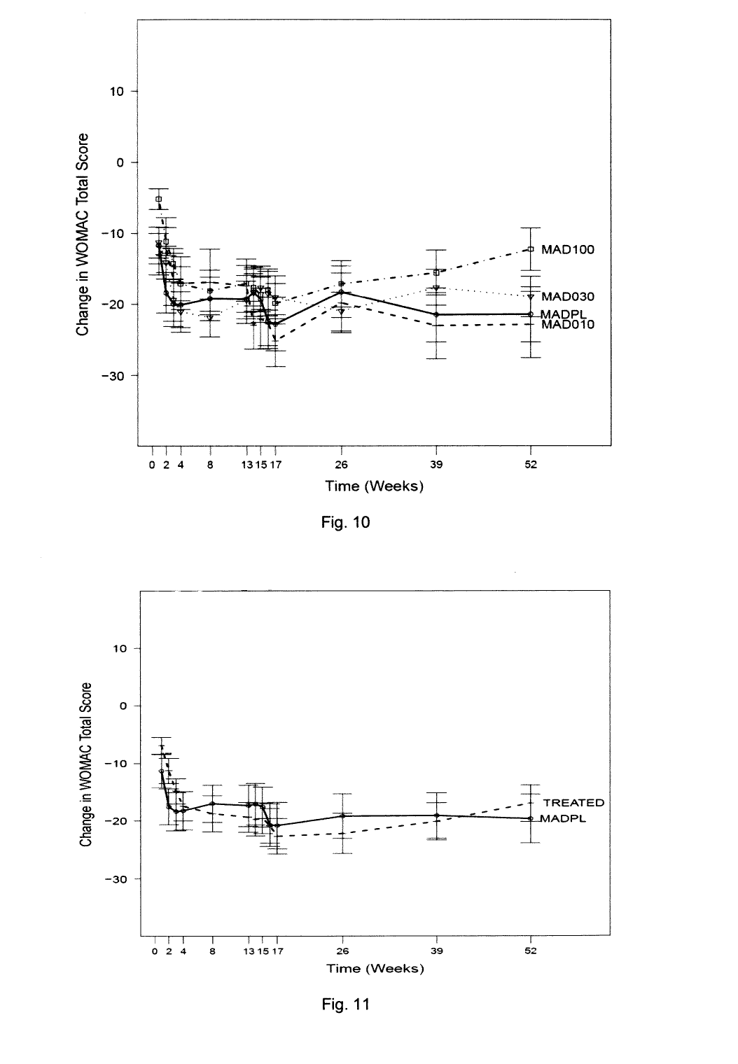

Without any genetic stratification, the clinical outcomes of the FGF18 therapy are the following: 1) significant increase in total cartilage volume in treated subjects (MAD100) compared to placebos (p-value=0.0157); 2) marginally smaller improvement in WOMAC total scores in treated subjects (MAD100) compared to placebos (p-value=0.1044); 3) 20% of AIRs in treated subjects. These results are summarized in Table 13 and detailed results are presented in Table 14 and Table 15. FIGS. 10 and 13 are also provided for data visualization. It is understood that FIGS. 10 to 15 do not correspond to the multivariate linear model used for the analyses. These figures are only provided to facilitate results interpretation.

The proposed diagnostic test (Table 4) aims at:
1. Identifying sensitives and treat them with the proposed FGF18 dose (e.g. 100 mcg)
2. Identifying super-sensitives and treat them with a lower FGF18 dose (e.g. 30 mcg)
3. Identifying non-sensitives and exclude them from FGF18 therapy.

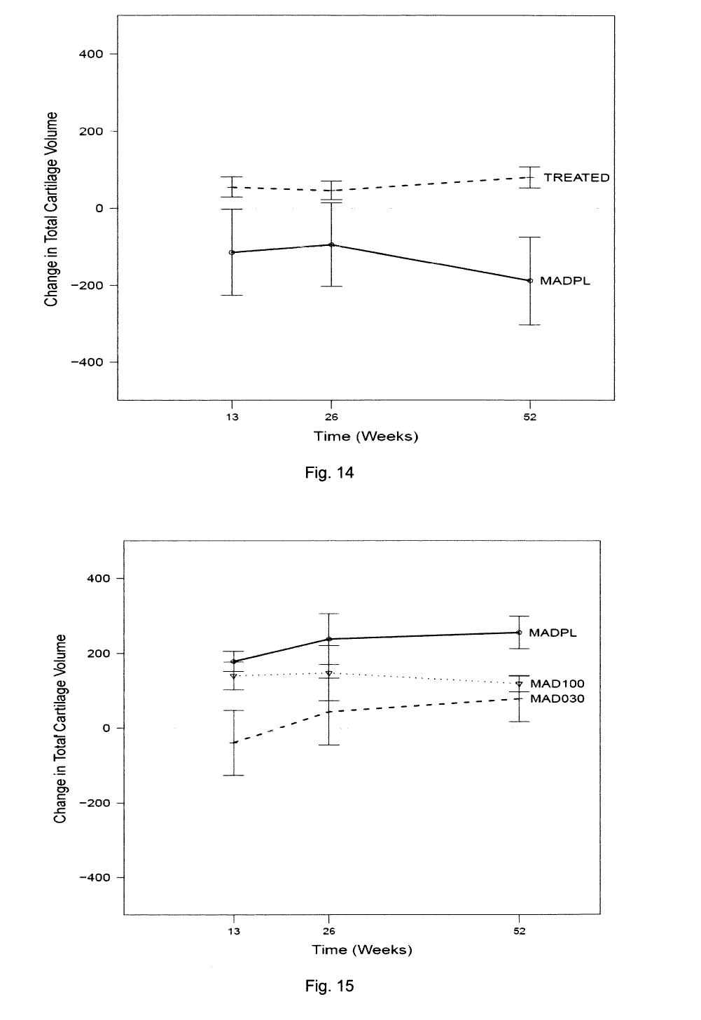

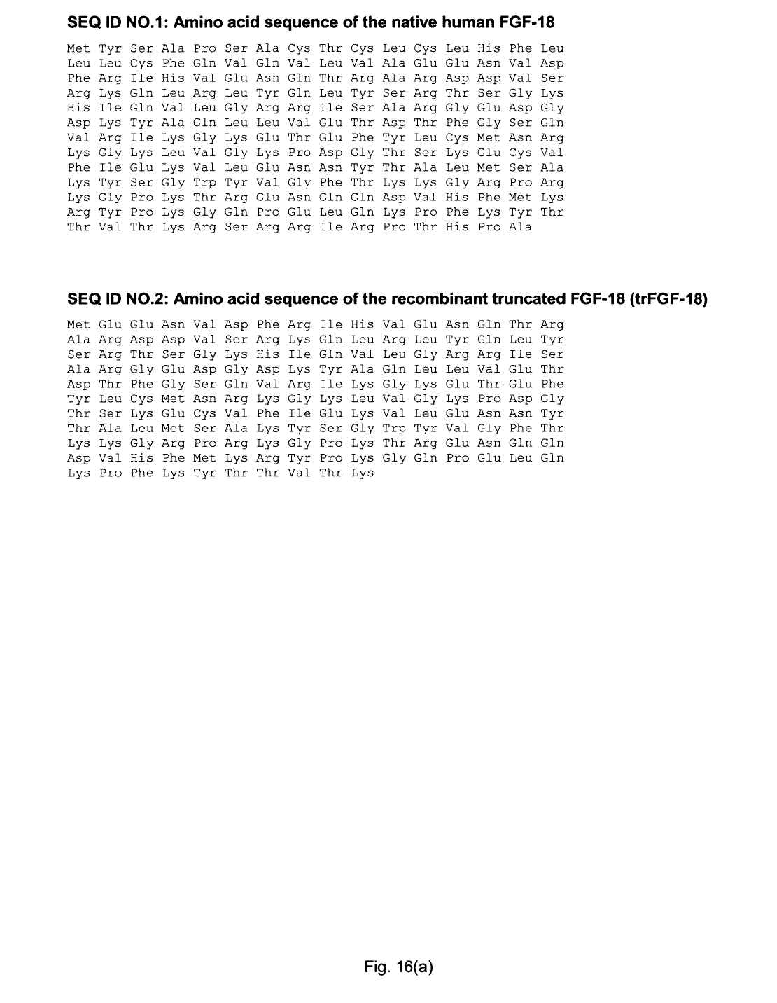

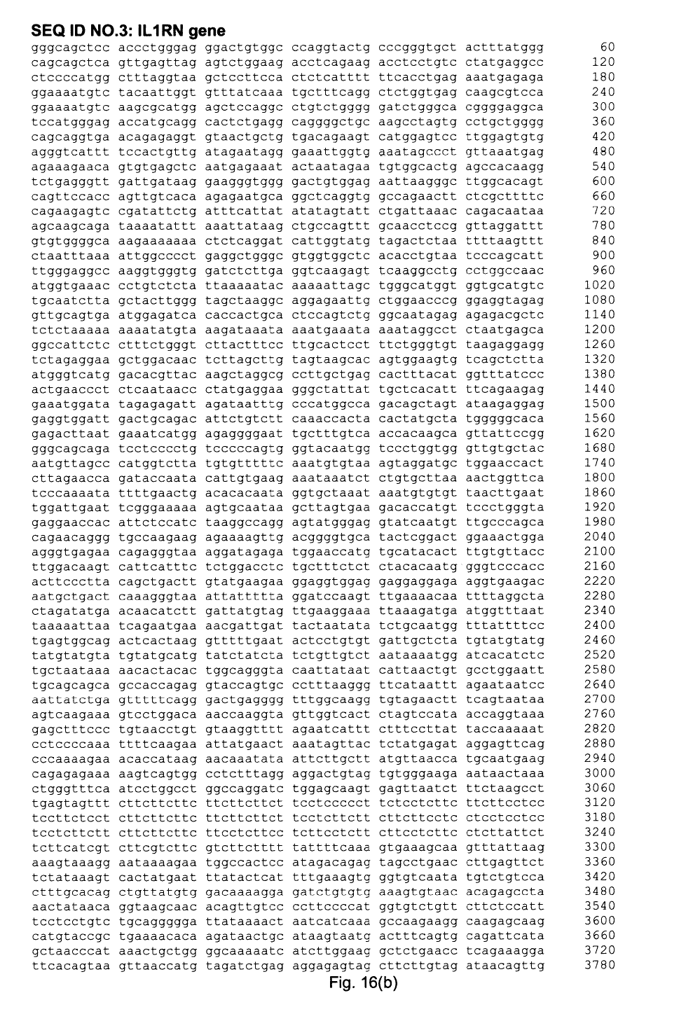

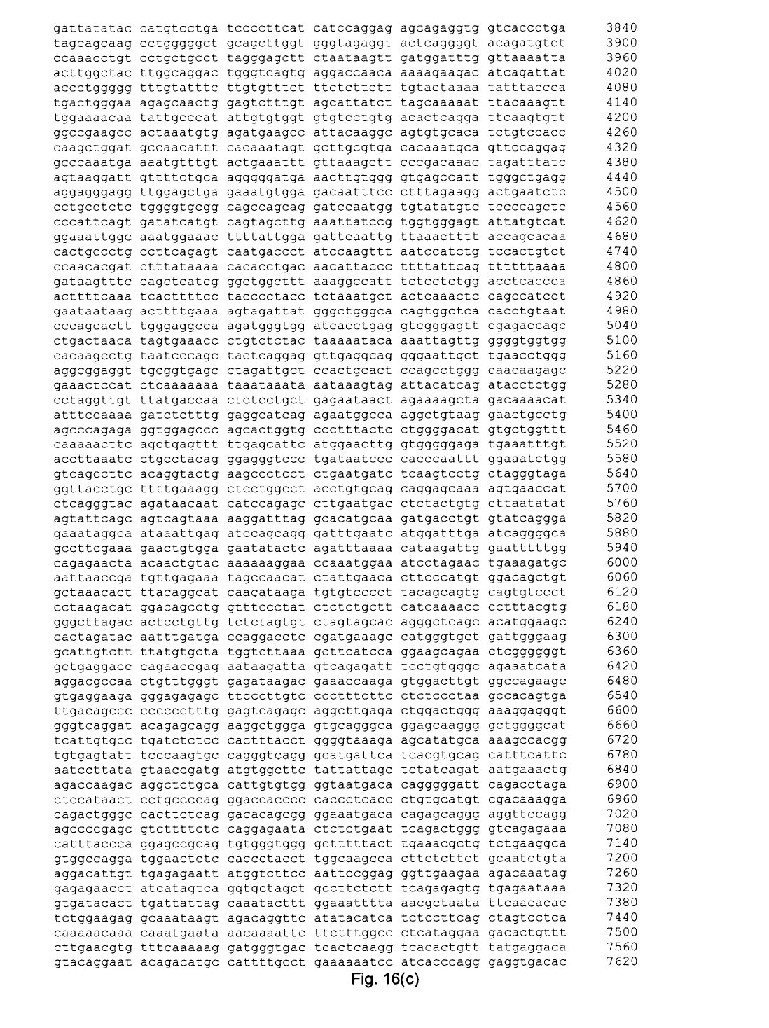

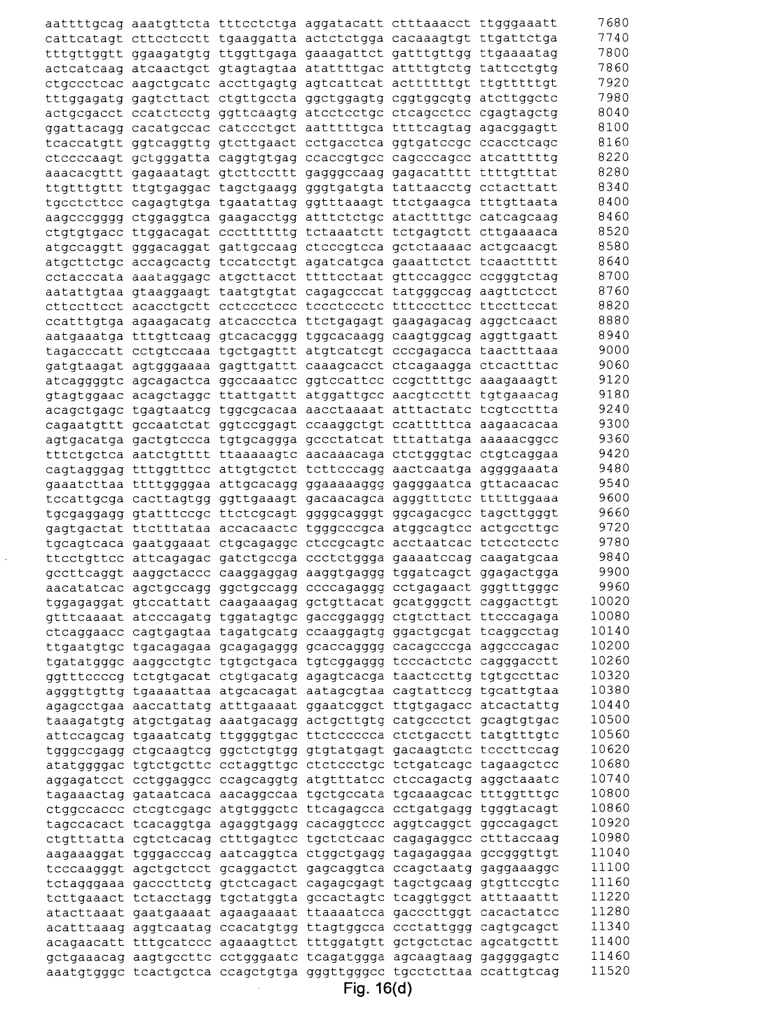

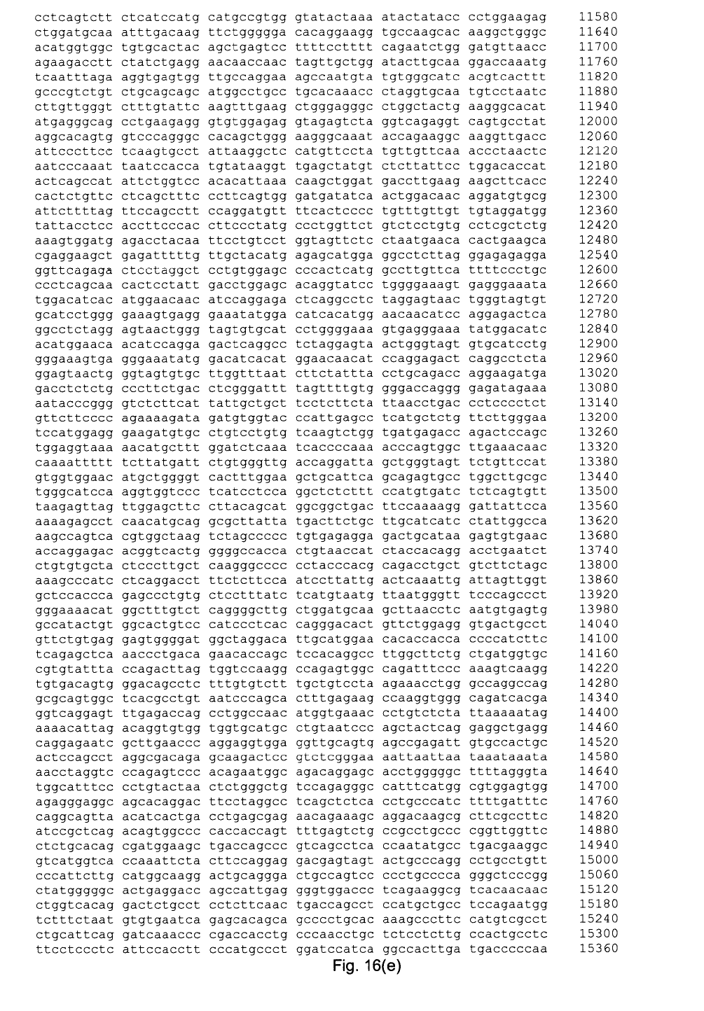

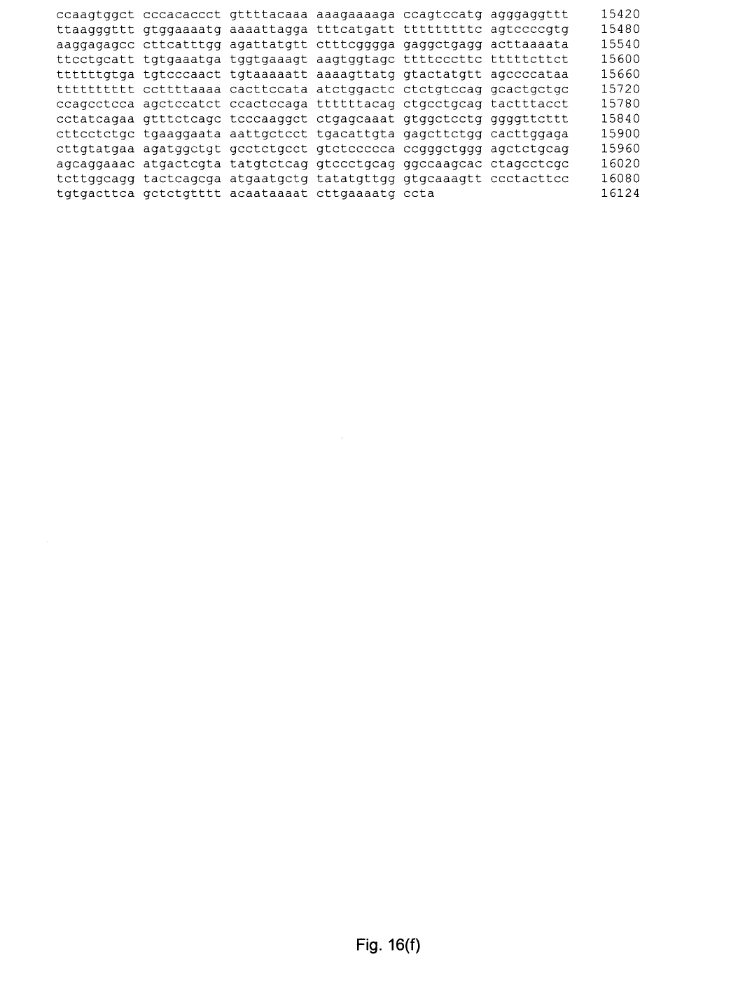

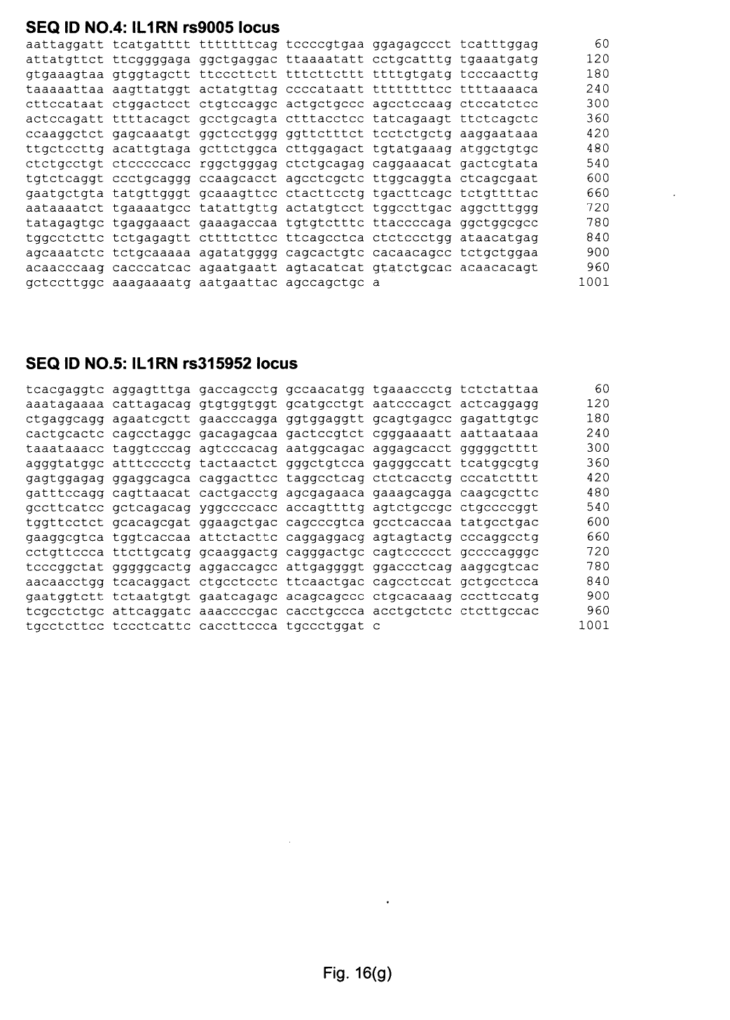

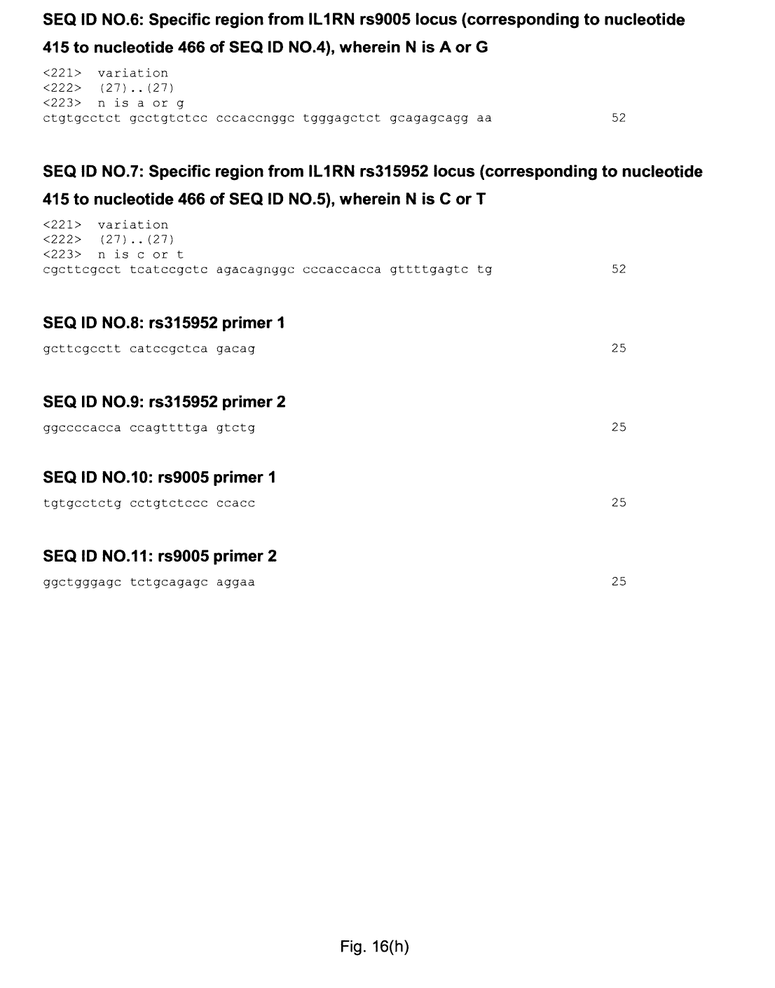

Retrospectively, the clinical outcomes, for subjects elected for FGF18 therapy, are
1. Significant increase in total cartilage volume in treated subjects (sensitives from MAD100 cohort+super-sensitives from MAD030 cohort) compared to matched placebos (p-value=0.0016 Table 18, FIG. 14). Simulation studies (bootstrap) showed that this cartilage volume improvement is significantly higher than the improvement obtained when no diagnostic test is used (p-value<1E−4)
2. Comparable improvement in WOMAC total scores between treated subjects and placebos (p-value=0.6603, Table 17, FIG. 11)
3. 11.43% of AIRs in treated subjects (Table 16)

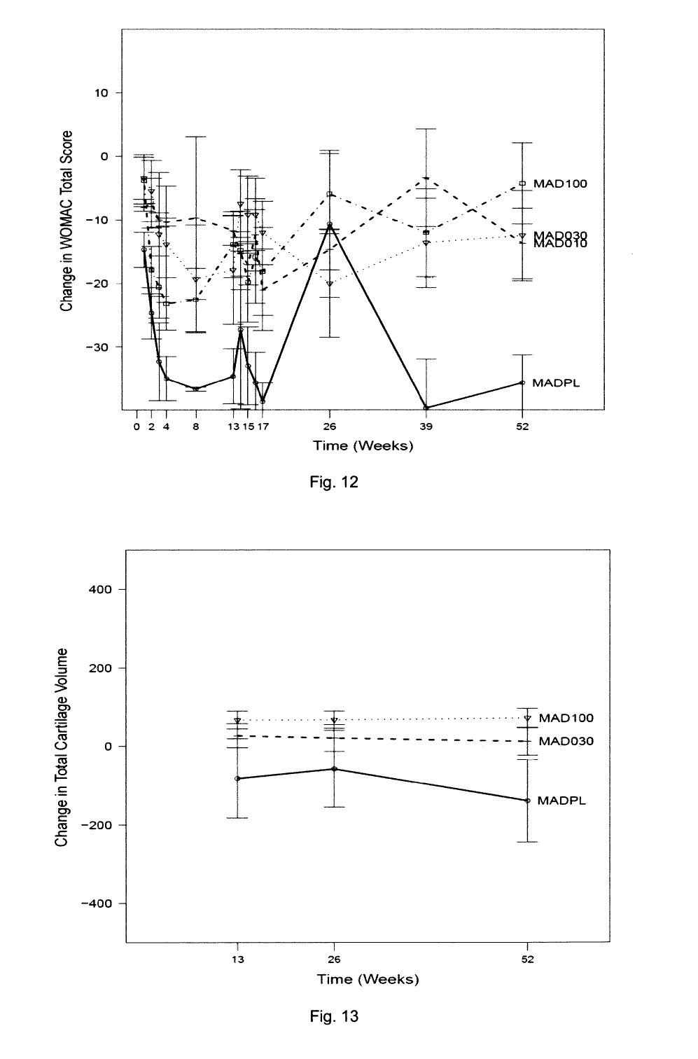

By contrast, subjects identified as non-sensitives have the following clinical outcomes:
1. Significantly lower improvement in total cartilage volume in treated subjects (non-sensitives from MAD100 cohort) compared to matched placebos (p-value=0.0289, Table 21) Subjects from the MAD030 cohort had similar outcome than subjects from the MAD100 cohort (FIG. 15). Thus none of the investigated dose showed an improvement with respect to placebos.
2. Although the p-value from the multivariate linear model is not significant (p-value=0.3068, Table 20), there is no improvement in WOMAC total score for treated subjects (median change=−1) while there is some improvement for placebos (median change=−39). Subjects from the MAD010 and MAD030 cohorts had similar outcome than subjects from the MAD100 cohort (FIG. 12). Thus none of the investigated dose showed an improvement with respect to placebos.
3. 22.22% of AIRs in treated subjects (Table 19)

It is anticipated that similar results will be achieved with other anabolic drugs such as BMP-2, BMP-7, GDF-5, FGFβ, FGF-8, FGF-9, SOX-9 enhancers, TGFβ, and any variants thereof.

### TABLES

TABLE 1
List of candidate SNPs

| Gene/description | Tested SNPs |
|---|---|
| FGF18 | rs3806929, rs4073716, rs9313543, rs4076077, rs4073717, rs6555956, |
| | rs10065728, rs4620037, rs11553493 |
| FGFR1 | rs2288696, rs2978073, rs11777067, rs6983315, rs7012413, rs6996321 |
| FGFR2 | rs3135810, rs2278202, rs1649200, rs7090018, rs2912759, rs2912787, |
| | rs2981449, rs2981432, rs10736303, rs1078806, rs2981575, rs1219648, |
| | rs1219643, rs2912774, rs2162540, rs2981582, rs3135715, rs3750819, |
| | rs755793 |
| FGFR3 | rs17880763, rs17881656, rs17882190, rs17884368 |
| FGFR4 | rs442856, rs422421, rs2011077 |
| FGFRL1 | rs4647934 |
| IL10 | rs1878672, rs3024493, rs1554286, rs3024491, rs3024490 |
| IL1A | rs1304037, rs3783550, rs3783525, rs1800587 |
| IL1B | rs1143627, rs1143634, rs1143633, rs3136558 |
| IL1RN | rs9005, rs315952, rs444413, rs3181052, rs419598, rs423904, |
| | rs442710, rs447713, rs451578, rs432014, rs431726, rs452204, |
| | rs3087266, rs579543 |
| IL6 | rs1800795, rs1800797, rs1474347, rs2069840, rs1800796 |
| marginal association with | rs5934659, rs12407610, rs1344049, rs10954969, rs1522844, |
| AIRs (from whole-genome | rs2685592, rs6697273, rs887071, rs1105227, rs6846033, rs871746, |
| scan) | rs11815080, rs6949763, rs897718, rs7651624, rs6989732, rs7786717, |
| | rs10093384, rs11737974, rs3122569, rs12453065, rs1992509, |
| | rs2202731, rs6897534, rs747159, rs4342357, rs2447011, rs4770271, |
| | rs10430746, rs7032155, rs10948190, rs7073333, rs6495812, rs946120, |
| | rs1047813, rs2032790, rs3865404, rs11040899, rs1968294, rs723077 |
| marginal association with | rs12410403, rs587505, rs9902708, rs734397, rs894013, rs932241 |
| WOMAC total score (from | |
| whole-genome scan) | |
| TNFRS1B | rs1061622 |
| VDR region | rs731236, rs7975232, rs1544410 |

TABLE 2a
Details of TaqMan SNP Id screened in period 1

| | | | NCBI | |
|---|---|---|---|---|
| Gene Symbol | rs Id | Assay Id | alleles | Assay type |
| FGF18 | rs4073716 | C_27537611_10 | C/T | Functionally Tested |
| FGF18 | rs11553493 | NA | G/T | Custom |
| FGF18, NPM1 | rs3806929 | C_11274941_10 | C/T | Functionally Tested |
| FGFR2 | rs755793 | C_2414603_10 | C/T | Validated |
| FGFR3 | rs17881656 | NA | C/T | Custom |
| FGFR3, LETM1 | rs17880763 | C_58182661_10 | A/T | Functionally Tested |
| FGFR3, LETM1 | rs17882190 | C_58182657_10 | A/G | Functionally Tested |
| IL1B | rs1143627 | C_1839944_10 | C/T | Validated |
| IL-6 | rs1800795 | hCV1839697 | C/G | Custom/SNPlex system |
| IL6, LOC541472 | rs1800796 | C_11326893_10 | C/G | Functionally Tested |
| LETM1, FGFR3 | rs17884368 | C_58182646_10 | A/G | Functionally Tested |
| LOC100131885, FGFR2 | rs3750819 | C_27511529_10 | C/G | Functionally Tested |
| LOC541472, IL6 | rs1800797 | C_1839695_20 | A/G | Functionally Tested |
| TNFRSF1B | rs1061622 | C_8861232_20 | G/T | Functionally Tested |
| VDR | rs7975232 | C_28977635_10 | A/C | Functionally Tested |
| VDR | rs731236 | C_2404008_10 | C/T | Functionally Tested |
| VDR | rs1544410 | C_8716062_10 | A/G | Validated |

TABLE 2b
Details of TaqMan SNP Id screened in period 2

| Gene | | | | |
|---|---|---|---|---|
| Symbol | rs Id | Assay Id | NCBI alleles | Assay type |
| IL1RN | rs9005 | C_3133528_10 | A/G | Functionally |
| | | | | Tested |
| IL1RN | rs315952 | C_11512470_10 | C/T | Validated |

TABLE 3
Taqman primer sequences

| | Applied | |
|---|---|---|
| SNP | Biosystems | |
| Reference | assay ID | Primer sequences |
| rs315952 | C_11512470_10 | Primer 1: GCTTCGCCTTCATCCGCTCAGACAG or complementary |
| | | sequence |
| | | Primer 2: GGCCCCACCACCAGTTTTGAGTCTG or complementary |
| | | sequence |
| rs9005 | C_3133528_10 | Primer 1: TGTGCCTCTGCCTGTCTCCCCCACC or complementary |
| | | sequence |
| | | Primer 2: GGCTGGGAGCTCTGCAGAGCAGGAA or complementary |
| | | sequence |

TABLE 4
Identified genotype categories in the Multiple Ascending Dose
cohort (100 mcg)

| | | rs9005 (A/G) | |
|---|---|---|---|
| | | GG | A carriers |
| rs315952 | TT | group A: | group B: |
| (T/C) | | non-sensitives | Sensitives |
| | | (20% of MAD100) | (27% of MAD100) |
| | C carriers | group C: | group D: |
| | | Sensitives | super-sensitives |
| | | (38% of MAD100) | (15% of MAD100) |

TABLE 5
Contingency table: AIR predictions based on rs9005 and rs315952
genotypes with subjects from the FGF18 MAD100 arm (n = 45)

| | | True AIR status | |
|---|---|---|---|
| | | Subjects | Subjects without |
| | | with AIRs | AIRs |
| Predicted status AIRs | Predicted with | 5 | 2 |
| | Predicted without | 4 | 34 |
| | AIRs | | |

TABLE 6
Performance at predicting AIRs based on rs9005 and rs315952 genotypes
with subjects from the FGF18 MAD100 arm (n = 45)

| Performance metrics | value |
|---|---|
| Sensitivity | 55.56% |
| Accuracy | 86.67% |
| Specificity | 94.44% |
| Precision | 71.43% |
| Negative predictive value | 89.47% |
| Sensitivity and precision (F1 score) | 62.50% |

TABLE 7
Contingency table: AIR predictions based on presence/absence of the
C-T-A haplotype with subjects from the FGF18 MAD100 arm (n = 48)

| | | True AIR status | |
|---|---|---|---|
| | | Subjects | Subjects without |
| | | with AIRs | AIRs |
| Predicted status | Predicted with AIRs | 6 | 7 |
| | Predicted without AIRs | 4 | 31 |

TABLE 8
Performance at predicting AIRs based on presence/absence of the C-T-A
haplotype with subjects from the FGF18 MAD100 arm (n = 48)

| Performance metrics | value |
|---|---|
| Sensitivity | 60% |
| Accuracy | 77.08% |
| Specificity | 81.58% |
| Precision | 46.15% |
| Negative predictive value | 88.57% |
| Sensitivity and precision (F1 score) | 52.17% |

TABLE 9
Multivariate linear modeling for change in total cartilage volume with
placebo subjects only

| | regression | | | |
|---|---|---|---|---|
| model term | coefficient | Standard Error | Z-score | p-value |
| Intercept | 78.44 | 23.68 | 3.31 | 0.0035 |
| group | 83.11 | 33.85 | 2.46 | 0.0234 |
| [B-C-D; A only] | | | | |
| Kellgren-Lawrence | −12.93 | 22.36 | −0.58 | 0.5695 |
| grade [2; 3] | | | | |
| Age [<65; >=65] | −15.83 | 20.86 | −0.76 | 0.4569 |
| BMI [<30; >=30] | 4.02 | 21.61 | 0.19 | 0.8545 |
| Gender [Female; Male] | −15.01 | 20.31 | −0.74 | 0.4683 |

TABLE 10
Multivariate linear modeling for change in WOMAC total score with
placebo subjects only

| | regression | | | |
|---|---|---|---|---|
| model term | coefficient | Standard Error | Z-score | p-value |
| Intercept | 63.76 | 20.40 | 3.13 | 0.0051 |
| group | −13.98 | 28.71 | −0.49 | 0.6313 |
| [B-C-D; A only] | | | | |
| Kellgren- | −25.47 | 18.83 | −1.35 | 0.1906 |
| Lawrence grade | | | | |
| [2; 3] | | | | |
| Age [<65; >=65] | 0.34 | 17.66 | 0.02 | 0.9847 |
| BMI [<30; >=30] | 46.97 | 18.08 | 2.60 | 0.0168 |
| Gender | 29.74 | 17.01 | 1.75 | 0.0950 |
| [Female; Male] | | | | |

TABLE 11
Contingency table: Kellgren-Lawrence grade (3 or 2) based on
presence/absence of the ‘rs9005 GG rs315952 TT’ genotype -
Analysis was performed using all subjects from all dose regimen
(including placebos). Fisher's exact test p-value is 0.0179, odds ratio
is 0.306 with 95% confidence interval [0.096, 0.885].

| genotype | Grade 3 | Grade 2 |
|---|---|---|
| rs9005 GG rs315952 TT | 7 | 15 |
| other | 60 | 39 |

TABLE 12
Contingency table: Kellgren-Lawrence grade (3 or 2) based on
presence/absence of the ‘rs9005 A carriers rs315952 C carriers’ genotype -
Analysis was performed using all subjects from all dose regimen
(including placebos). Fisher's exact test p-value is 0.2736, odds ratio is
1.693 with 95% confidence interval [0.637, 4.769].

| genotype | Grade 3 | Grade 2 |
|---|---|---|
| rs9005 A carriers rs315952 C | 17 | 9 |
| carriers | | |
| other | 50 | 45 |

TABLE 13
Clinical outcome without diagnostic test (45 subjects treated with FGF18
100 mcg and 27 placebos) - Delta corresponds to the difference between
the median change in placebos and the median change in treated
subjects. P-value corresponds to the p-value from a multivariate linear
model adjusting for gender, age, BMI and KL grade.

| | | median change in | | |
|---|---|---|---|---|
| Groups | median change in | treated subjects | | |
| A, B, C, D | placebos | (MAD100) | delta | p-value |
| Change in | −19 | -10 | 9 | 0.1044 |
| WOMAC | | | | |
| total score | | | | |
| Change in | −44.68 | 102.25 | 146.93 | 0.0157 |
| total cartilage | | | | |
| volume | | | | |
| % AIRs | 3.7 | 20 | 16.3 | NA |

TABLE 14
Multivariate linear modeling for change in WOMAC total
score with all placebos and all MAD100 treated subjects

| | regression | Standard | | p-value | LR Chi- | p-value |
|---|---|---|---|---|---|---|
| model term | coefficient | Error | Z-score | (GLM) | square (anova) | (anova) |
| Intercept | 59.51 | 14.86 | 4.00 | 0.0002 | NA | NA |
| Age [<65; >=65] | 11.07 | 12.61 | 0.88 | 0.3834 | 0.77 | 0.3802 |
| Arm (dose 100 mcg) | 19.51 | 12.02 | 1.62 | 0.1091 | 2.64 | 0.1044 |
| BMI [<30; >=30] | 13.24 | 12.09 | 1.10 | 0.2774 | 1.20 | 0.2734 |
| Gender [Female; Male] | 20.13 | 11.84 | 1.70 | 0.0937 | 2.89 | 0.0890 |
| Kellgren-Lawrence grade [2; 3] | 0.14 | 11.79 | 0.01 | 0.9902 | 0.00 | 0.9902 |

TABLE 15
Multivariate linear modeling for change in total cartilage
volume with all placebos and all MAD100 treated subjects

| | regression | Standard | | p-value | LR Chi- | p-value |
|---|---|---|---|---|---|---|
| model term | coefficient | Error | Z-score | (GLM) | square (anova) | (anova) |
| Intercept | 79.10 | 13.37 | 5.92 | 0.0000 | NA | NA |
| Age [<65; >=65] | −15.10 | 11.35 | −1.33 | 0.1878 | 1.77 | 0.1832 |
| Arm [placebos; treated] | 26.12 | 10.81 | 2.42 | 0.0185 | 5.84 | 0.0157 |
| BMI [<30; >=30] | −14.60 | 10.87 | −1.34 | 0.1841 | 1.80 | 0.1795 |
| Gender [Female; Male] | 1.05 | 10.65 | 0.10 | 0.9216 | 0.01 | 0.9213 |
| Kellgren-Lawrence grade [2; 3] | −1.25 | 10.61 | −0.12 | 0.9065 | 0.01 | 0.9061 |

TABLE 16
Clinical outcome for subjects classified as 1) sensitives (groups B and C,
n = 29, treated with FGF18 100 mcg) or 2) super-sensitives (group D,
n = 6, treated with a lower FGF18 dose: 30 mcg). 24 placebos, with
genotypes from either group B, C or D, were included in the analysis -
Delta corresponds to the difference between the median change in
placebos and the median change in treated subjects. P-value corresponds
to the p-value from a multivariate linear model adjusting for gender, age,
BMI and KL grade.

| | median | median change in | | |
|---|---|---|---|---|
| | change | treated subjects | | |
| Groups B, C, D | in placebos | (MAD100 + MAD30) | delta | p-value |
| Change in | −16.5 | −13 | 3.5 | 0.6603 |
| WOMAC | | | | |
| total | | | | |
| score | | | | |
| Change in | −114.91 | 102.25 | 217.16 | 0.0016 |
| total cartilage | | | | |
| volume | | | | |
| % AIRs | 0 | 11.43 | 11.43 | NA |

TABLE 17
Multivariate linear modeling for change in WOMAC total score with subjects classified
as 1) sensitives (groups B and C, n = 29, treated with FGF18 100 mcg) or 2)
super-sensitives (group D, n = 6, treated with a lower FGF18 dose: 30 mcg).
24 placebos, with genotypes from either group B, C or D, were included in the analysis.

| | regression | Standard | | p-value | LR Chi- | p-value |
|---|---|---|---|---|---|---|
| model term | coefficient | Error | Z-score | (GLM) | square (anova) | (anova) |
| Intercept | 67.09 | 16.25 | 4.13 | 0.0001 | NA | NA |
| Age [<65; >=65] | 7.23 | 13.33 | 0.54 | 0.5900 | 0.29 | 0.5877 |
| Arm [placebos; treated] | 5.82 | 13.24 | 0.44 | 0.6621 | 0.19 | 0.6603 |
| BMI [<30; >=30] | 3.87 | 12.82 | 0.30 | 0.7641 | 0.09 | 0.7629 |
| Gender [Female; Male] | 16.54 | 13.68 | 1.21 | 0.2322 | 1.46 | 0.2268 |
| Kellgren-Lawrence grade [2; 3] | 6.67 | 13.09 | 0.51 | 0.6124 | 0.26 | 0.6103 |

TABLE 18
Multivariate linear modeling for change in total cartilage volume with subjects classified
as 1) sensitives (groups B and C, n = 29, treated with FGF18 100 mcg) or 2) super-
sensitives (group D, n = 6, treated with a lower FGF18 dose: 30 mcg). 24 placebos,
with genotypes from either group B, C or D, were included in the analysis.

| | regression | Standard | | p-value | LR Chi- | p-value |
|---|---|---|---|---|---|---|
| model term | coefficient | Error | Z-score | (GLM) | square (anova) | (anova) |
| Intercept | 64.94 | 14.46 | 4.49 | 0.0000 | NA | NA |
| Age [<65; >=65] | −15.89 | 11.86 | −1.34 | 0.1860 | 1.79 | 0.1803 |
| Arm [placebos; treated] | 37.14 | 11.78 | 3.15 | 0.0027 | 9.94 | 0.0016 |
| BMI [<30; >=30] | −5.47 | 11.40 | −0.48 | 0.6332 | 0.23 | 0.6312 |
| Gender [Female; Male] | 1.14 | 12.18 | 0.09 | 0.9258 | 0.01 | 0.9254 |
| Kellgren-Lawrence grade [2; 3] | 1.27 | 11.65 | 0.11 | 0.9137 | 0.01 | 0.9133 |

TABLE 19
Clinical outcome for subjects classified as non-sensitives by the diagnostic
test (MAD100 n = 9, MADPL n = 3) - Delta corresponds to the
difference between the median change in placebos and the median change
in treated subjects. P-value corresponds to the p-value from a
multivariate linear model adjusting for gender, age, BMI and KL grade.

| | | median change in | | |
|---|---|---|---|---|
| | median change in | treated subjects | | |
| Group A only | placebos | (MAD100) | delta | p-value |
| Change in | −39 | −1 | 38 | 0.3068 |
| WOMAC | | | | |
| total score | | | | |
| Change in total | 224.56 | 117.92 | −106.64 | 0.0289 |
| cartilage | | | | |
| volume | | | | |
| % AIRs | 33.33 | 22.22 | −11.11 | NA |

TABLE 20
Multivariate linear modeling for change in WOMAC total score, with subjects classified
as non-sensitives by the diagnostic test (MAD100 n = 9, MADPL n = 3)

| | regression | Standard | | p-value | LR Chi- | p-value |
|---|---|---|---|---|---|---|
| model term | coefficient | Error | Z-score | (GLM) | square (anova) | (anova) |
| Intercept | 38.99 | 40.18 | 0.97 | 0.3693 | NA | NA |
| Age [<65; >=65] | 1.62 | 45.92 | 0.04 | 0.9730 | 0.00 | 0.9718 |
| Arm [placebos; treated] | 43.10 | 42.18 | 1.02 | 0.3462 | 1.04 | 0.3068 |
| BMI [<30; >=30] | 18.24 | 38.93 | 0.47 | 0.6558 | 0.22 | 0.6393 |
| Gender [Female; Male] | 43.01 | 33.47 | 1.29 | 0.2461 | 1.65 | 0.1987 |
| Kellgren-Lawrence grade [2; 3] | −19.53 | 34.44 | −0.57 | 0.5911 | 0.32 | 0.5706 |

TABLE 21
Multivariate linear modeling for change in total cartilage volume with subjects
classified as non-sensitives by the diagnostic test (MAD100 n = 9, MADPL n = 3)

| | regression | Standard | | p-value | LR Chi- | p-value |
|---|---|---|---|---|---|---|
| model term | coefficient | Error | Z-score | (GLM) | square (anova) | (anova) |
| Intercept | 128.67 | 15.26 | 8.43 | 0.0002 | NA | NA |
| Age [<65; >=65] | 47.00 | 17.44 | 2.70 | 0.0358 | 7.27 | 0.0070 |
| Arm [placebos; treated] | −35.00 | 16.02 | −2.19 | 0.0715 | 4.78 | 0.0289 |
| BMI [<30; >=30] | 30.67 | 14.78 | 2.07 | 0.0834 | 4.30 | 0.0380 |
| Gender [Female; Male] | −39.00 | 12.71 | −3.07 | 0.0220 | 9.42 | 0.0022 |
| Kellgren-Lawrence grade [2; 3] | 7.00 | 13.08 | 0.54 | 0.6117 | 0.29 | 0.5925 |

TABLE 22
Summary of clinical outcome and potential therapeutic options based on
rs9005 and rs315952 genotypes

| | Group A | Groups B & C | Group D | |
|---|---|---|---|---|
| | 100 mcg | 100 mcg | 100 mcg | 30 mcg |
| Change in | Significant | Change in | Change in | Change in |
| WOMAC total | WOMAC | WOMAC | WOMAC higher | WOMAC |
| score | worsening | comparable to | than placebo | comparable to |
| | compared to | placebo | | placebo |
| | placebo | | | |
| Change in total | No improvement | Significant | Significant | Highest cartilage |
| cartilage volume | | cartilage volume | cartilage volume | volume |
| | | improvement | improvement | improvement |
| | | | (highest gain | (significantly |
| | | | among all groups | better than 100 mcg) |
| | | | treated at | |
| | | | 100 mcg) | |
| AIRs | 2/9 treated | 2/29 treated | 5/7 treated | 2/6 treated |
| | subjects (1/3 in | subjects (0/17 in | subjects (0/7 in | subjects (0/7 in |
| | placebos) | placebos) | placebos) | placebos) |

| | | | |
|---|---|---|---|
| Potential | Do not benefit | Treat up to | Treat at 30 mcg |
| therapeutic | from FGF18 | 100 mcg | |
| option | therapy | | |

### REFERENCES

1) WO2008/023063
2) WO2004/032849
3) WO2006/063362
4) WO2009/135218
5) WO 92/15712
6) U.S. Pat. No. 5,679,524
7) WO 91/02087
8) WO 90/09455
9) WO 95/17676
10) U.S. Pat. No. 5,302,509
11) U.S. Pat. No. 5,945,283
12) U.S. Pat. No. 5,605,798
13) WO 89/10414
14) http://www.cartilage.org/_files/contentmanagement/ICRS_evaluation.pdf
15) Lotz, 2010, Arthritis research therapy, 12:211
16) Ellsworth et al., 2002, Osteoarthritis and Cartilage, 10: 308-320
17) Shimoaka et al., 2002, J. Bio. Chem. 277(9):7493-7500
18) The Merck Manual, 17^{th} edition, page 449
19) Bellamy et al., 1988, J. Rheumatology, 15:1833-1840
20) Wolfe, 1999, Rheumatology, 38:355-361
21) Attur et al., Ann. Rheum. Dis. 2010, 69:856-861
22) Li et al., 2010, Genet Epidemiol 34:816-834
23) Benjamini and Hochberg, 1995, J. of the Royal Statistical Society Series B(57):289
24) Wirapati et al., 2011, Ann. Hum. Genet. 75(1):133-45
25) Bateson W. and Mendel G., 1909, “G. Mendel's principles of heredity”. Cambridge University
Press; 1909. Available: http://archive.org/details/mendelsprinciple00bate
26) Phillips P C., 1998, Genetics. 7; 149(3):1167-71.
27) Cordell H J., 2002, Hum. Mol. Genet. 11(20):2463-8.
28) Fisher R A, 1918, “The Correlation Between Relatives on the Supposition of Mendelian Inheritance”. Available: http://digital.library.adelaide.edu.au/dspace/handle/2440/15097.
29) Browning S R and Browning B L, 2011, Nature Reviews Genetics. 12(10):703-14.

1-18. (canceled)

19. A method of prognosing disorder severity in a subject having a cartilage disorder, the method comprising the steps of:
a) determining, from a nucleic acid sample, the genotype at both IL-1 RN rs9005 and IL-1RN rs315952; and
b) prognosing from the result of step a disorder severity.

20. The method according to claim 19, comprising the steps of:
a) determining, from the nucleic acid sample, the presence of the genotype G/G at IL-1RN rs9005 and T/T at IL-1RN rs315952; and
b) prognosing from the result of step a less severe form of cartilage disorder.

21. A method of predicting sensitivity to a drug prior to drug administration in a subject having a cartilage disorder, the method comprising the steps of:
a) determining, from a nucleic acid sample, the genotype at both loci IL-1 RN rs9005 and IL-1RN rs315952; and
b) predicting from the result of step a. high, intermediate, low or no sensitivity of said subject to said drug.

22. The method according to claim 21, comprising the steps of:
a) determining, from the nucleic acid sample, the presence of the genotype G/G at IL-1RN rs9005 and T/T at IL-1RN rs315952; and
b) predicting from the presence of said genotype low or no sensitivity to said drug.

23. The method according to claim 21, comprising the steps of:
a) determining, from the nucleic acid sample, the presence of the genotype(s) selected from the group consisting of:
i) IL-1RN rs9005 G/G and IL-1RN rs315952 TIC or C/C; or
ii) IL-1RN rs9005 A/G or A/A and IL-1RN rs315952 T/T; and
b) predicting from the presence of said genotype intermediate sensitivity to said drug.

24. The method according to claim 21, comprising the steps of:
a) determining, from the nucleic acid sample, the presence of the genotype A/A or A/G at IL-1RN rs9005 and C/C or C/T at IL-1RN rs315952; and
b) predicting from the presence of said genotype high sensitivity to said drug.

25. A method for selecting a patient having a cartilage disorder for inclusion in or exclusion from treatment, or clinical trial, with a drug, based on the likelihood of his/her sensitivity to said drug, comprising the steps of:
a) determining, from a nucleic acid sample, the genotype at both loci IL-1RN rs9005 and IL-1RN rs315952, wherein the patient's genotype with respect to said loci is predictive about the patient's risk for being sensitive or not sensitive to said drug; and
b) selecting the sensitive patients as being suitable for treatment, or clinical trial, with said drug.

26. The method according to claim 25, comprising the steps of:
a) determining, from the nucleic acid sample, the presence of the genotype IL-1RN rs9005 G/G and IL-1RN rs315952 T/T; and
b) excluding the patient presenting said genotype from the treatment, or clinical trial, with the drug.

27. The method according to claim 25, comprising the steps of:
a) determining, from the nucleic acid sample, the presence of the genotype(s) selected from the group consisting of:
i) IL-1RN rs9005 G/G and IL-1RN rs315952 T/C or C/C; or
ii) IL-1RN rs9005 A/G or A/A and IL-1RN rs315952 T/T, T/C or C/C; and
b) including the patient presenting said genotype in the treatment, or clinical trial, with the drug.

28. A method for selecting a patient having a cartilage disorder for an alternative therapeutic regimen with a drug, based on the likelihood of said patient of being super-sensitive to said drug, comprising the steps of:
a) determining, from a nucleic acid sample, the genotype at both loci IL-1RN rs9005 and IL-1RN rs315952, wherein the patient's genotype with respect to said loci is predictive about the subject's risk for being super-sensitive to said drug; and
b) selecting said patient for an alternative therapeutic regimen, in which alternative therapeutic regimen the dose of the drug that is to be administered is reduced compared to the dose of said drug to be administered to a patient who does not present a risk for being super-sensitive to said drug.

29. A method for selecting a patient having a cartilage disorder for an alternative therapeutic regimen with a drug, based on the likelihood of said patient of having Acute Inflammatory Reaction (AIR) events when treated with said drug, the method comprising the steps of:
a) determining, from a nucleic acid sample, the genotype at both loci IL-1 RN rs9005 and IL-1RN rs315952, wherein the patient's genotype with respect to said loci is predictive about the subject's risk for developing AIR events in response to treatment with said drug; and
b) selecting said patient for an alternative therapeutic regimen, in which alternative therapeutic regimen the dose of drug that is to be administered is reduced compared to the dose of drug to be administered to a patient who does not present a risk for developing AIR events.

30. The method according to claim 28, comprising the steps of:
a) determining, from the nucleic acid sample, the presence of the genotype IL—A/G or A/A at IL-1RN rs9005 and T/C or C/C at IL-1RN rs315952; and
b) selecting the patient having said genotype for an alternative therapeutic regimen in which the dose of drug to be administered is reduced.

31. The method according to claim 29, comprising the steps of:
a) determining, from the nucleic acid sample, the presence of the genotype IL—A/G or A/A at IL-1RN rs9005 and T/C or C/C at IL-1RN rs315952; and
b) selecting the patient having said genotype for an alternative therapeutic regimen in which the dose of drug to be administered is reduced.

32. A kit comprising means for performing the method according to claim 19 and instructions for use.

33. The kit according to claim 32, comprising at least a couple of specific primers or probes for detecting the presence or absence of the alleles in rs9005 and rs315952.

34. The method according to claim 19, wherein the cartilage disorder is selected from the group consisting of osteoarthritis, cartilage injury, fractures affecting joint cartilage or surgical procedures with impact on joint cartilage, such as Microfracture.

35. The method according to claim 21, wherein the drug is a drug having anabolic effect on cartilage.

36. The method according to claim 35, wherein the drug having anabolic effect on cartilage is selected from the group consisting of FGF-18, BMP-2, BMP-7, GDF-5, FGFβ, FGF-8, FGF-9, SOX-9 enhancers or TGFβ and any variants thereof.

37. The method according to claim 36, wherein said drug is an FGF-18 such as sprifermin.


SEQUENCE LISTING (11 sequences)

SEQ ID NO: 1 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 2 - protein, 170 aa, artificial sequence
  Truncated FGF-18
MEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTK

SEQ ID NO: 3 - DNA, 16124 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttaggtaagctccttccactctcattttttcacctgagaaatgagagaggaaaatgtctacaattggtgtttatcaaatgctttcaggctctggtgagcaagcgtccaggaaaatgtcaagcgcatggagctccaggcctgtctgggggatctgggcacggggaggcatccatgggagaccatgcaggcactctgaggcaggggctgcaagcctagtgcctgctggggcagcaggtgaacagagaggtgtaactgctgtgacagaagtcatggagtccttggagtgtgagggtcattttccactgttgatagaatagggaaattggtgaaatagccctgttaaatgagagaaagaacagtgtgagctcaatgagaaatactaatagaatgtggcactgagccacaaggtctgagggttgattgataaggaagggtggggactgtggagaattaagggcttggcacagtcagttccaccagttgtcacaagagaatgcaggctcaggtggccagaacttctcgcttttccagaagagtccgatattctgatttcattatatatagtattctgattaaaccagacaataaagcaagcagataaaatatttaaattataagctgccagtttgcaacctccggttaggatttgtgtggggcaaagaaaaaaactctcaggatcattggtatgtagactctaattttaagtttctaatttaaaattggcccctgaggctgggcgtggtggctcacacctgtaatcccagcattttgggaggccaaggtgggtggatctcttgaggtcaagagttcaaggcctgcctggccaacatggtgaaaccctgtctctattaaaaatacaaaaattagctgggcatggtggtgcatgtctgcaatcttagctacttgggtagctaaggcaggagaattgctggaacccgggaggtagaggttgcagtgaatggagatcacaccactgcactccagtctgggcaatagagagagacgctctctctaaaaaaaaatatgtaaagataaataaaatgaaataaaataggcctctaatgagcaggccattctcctttctgggtcttactttccttgcactcctttctgggtgttaagaggaggtctagaggaagctggacaactcttagcttgtagtaagcacagtggaagtgtcagctcttaatgggtcatggacacgttacaagctaggcgccttgctgagcactttacatggtttatcccactgaaccctctcaataaccctatgaggaagggctattattgctcacattttcagaagaggaaatggatatagagagattagataatttgcccatggccagacagctagtataagaggaggaggtggattgactgcagacattctgtcttcaaaccactacactatgctatgggggcacagagacttaatgaaatcatggagaggggaattgctttgtcaaccacaagcagttattccgggggcagcagatcctcccctgtcccccagtgggtacaatggtccctggtgggttgtgctacaatgttagcccatggtcttatgtgtttttcaaatgtgtaaagtaggatgctggaaccactcttagaaccagataccaatacattgtgaagaaataaatctctgtgcttaaaactggttcatcccaaaatattttgaactgacacacaataggtgctaaataaatgtgtgttaacttgaattggattgaattcgggaaaaaagtgcaataagcttagtgaagacaccatgttccctgggtagaggaaccacattctccatctaaggccaggagtatgggaggtatcaatgtttgcccagcacagaacagggtgccaagaagagaaaagttgacggggtgcatactcggactggaaactggaagggtgagaacagagggtaaaggatagagatggaaccatgtgcatacactttgtgttaccttggacaagtcattcatttctctggacctctgctttctctctacacaatggggtcccaccacttcccttacagctgacttgtatgaagaaggaggtggaggaggaggagaaggtgaagacaatgctgactcaaagggtaaattatttttaggatccaagtttgaaaacaattttaggctactagatatgaacaacatcttgattatgtagttgaaggaaattaaagatgaatggtttaattaaaaattaatcagaatgaaaacgattgattactaatatatctgcaatggtttattttcctgagtggcagactcactaaggtttttgaatactcctgtgtgattgctctatgtatgtatgtatgtatgtatgtatgcatgtatctatctatctgttgtctaataaaatggatcacatctctgctaataaaaacactacactggcagggtacaattataatcattaactgtgcctggaatttgcagcagcagccaccagaggtaccagtgccctttaagggttcataatttagaataatccaattatctgagtttttcagggactgaggggtttggcaaggtgtagaactttcagtaataaagtcaagaaagtcctggacaaaccaaggtagttggtcactctagtccataaccaggtaaagagctttccctgtaacctgtgtaaggttttagaatcatttctttccttattaccaaaaatcctccccaaattttcaagaaattatgaactaaatagttactctatgagataggagttcagcccaaaagaaacaccataagaacaaatataattcttgcttatgttaaccatgcaatgaagcagagagaaaaagtcagtggcctctttaggaggactgtagtgtgggaagaaataactaaactgggtttcaatcctggcctggccaggatctggagcaagtgagttaatctttctaagccttgagtagtttcttcttcttcttcttcttcttcctcccccttctcctcttcttcttcctcctccttctcctcttcttcttcttcttcttcttcctcttcttcttcttcctcctcctcctcctcctcttcttcttcttcttcttcctcttcctcttcctcttcttcctcttcctcttattcttcttcatcgtcttcgtcttcgtcttctttttattttcaaagtgaaagcaagtttattaagaaagtaaaggaataaaagaatggccactccatagacagagtagcctgaaccttgagttcttctataaagtcactatgaatttatactcattttgaaagtgggtgtcaatatgtctgtccactttgcacagctgttatgtggacaaaaggagatctgtgtgaaagtgtaacacagagcctaaactataacaggtaagcaacacagttgtccccttccccatggtgtctgttcttctccatttcctcctgtctgcagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacagtaagttaaccatgtagatctgagaggagagtagcttcttgtagataacagttggattatataccatgtcctgatccccttcatcatccaggagagcagaggtggtcaccctgatagcagcaagcctgggggctgcagcttggtgggtagaggtactcaggggtacagatgtctccaaacctgtcctgctgccttagggagcttctaataagttgatggatttggttaaaattaacttggctacttggcaggactgggtcagtgaggaccaacaaaaagaagacatcagattataccctgggggtttgtatttcttgtgtttctttctcttctttgtactaaaatatttacccatgactgggaaagagcaactggagtctttgtagcattatcttagcaaaaatttacaaagtttggaaaacaatattgcccatattgtgtggtgtgtcctgtgacactcaggattcaagtgttggccgaagccactaaatgtgagatgaagccattacaaggcagtgtgcacatctgtccacccaagctggatgccaacatttcacaaatagtgcttgcgtgacacaaatgcagttccaggaggcccaaatgaaaatgtttgtactgaaatttgttaaagcttcccgacaaactagatttatcagtaaggattgttttctgcaagggggatgaaacttgtggggtgagccatttgggctgaggaggagggaggttggagctgagaaatgtggagacaatttccctttagaaggactgaatctccctgcctctctggggtgcggcagccagcaggatccaatggtgtatatgtctccccagctccccattcagtgatatcatgtcagtagcttgaaattatccgtggtgggagtattatgtcatggaaattggcaaatggaaacttttattggagattcaattgttaaacttttaccagcacaacactgccctgccttcagagtcaatgaccctatccaagtttaatccatctgtccactgtctccaacacgatctttataaaacacacctgacaacattacccttttattcagttttttaaaagataagtttccagctcatcgggctggctttaaaggccatttctcctctggacctcacccaacttttcaaatcacttttcctacccctacctctaaatgctactcaaactccagccatcctgaataataagacttttgaaaagtagattatgggctgggcacagtggctcacacctgtaatcccagcactttgggaggccaagatgggtggatcacctgaggtcgggagttcgagaccagcctgactaacatagtgaaaccctgtctctactaaaaatacaaaattagttgggggtggtggcacaagcctgtaatcccagctactcaggaggttgaggcaggggaattgcttgaacctgggaggcggaggttgcggtgagcctagattgctccactgcactccagcctgggcaacaagagcgaaactccatctcaaaaaaataaataaataaataaagtagattacatcagatacctctggcctaggttgtttatgaccaactctcctgctgagaataactagaaaagctagacaaaacatatttccaaaagatctctttggaggcatcagagaatggccaaggctgtaaggaactgcctgagcccagagaggtggagcccagcactggtgccctttactcctggggacatgtgctggtttcaaaaacttcagctgagtttttgagcattcatggaacttggtgggggagatgaaatttgtaccttaaatcctgcctacagggagggtccctgataatccccacccaatttggaaatctgggtcagccttcacaggtactgaagccctcctctgaatgatctcaagtcctgctagggtagaggttacctgcttttgaaaggctcctggcctacctgtgcagcaggagcaaaagtgaaccatctcagggtacagataacaatcatccagagccttgaatgacctctactgtgcttaatatatagtattcagcagtcagtaaaaaggatttaggcacatgcaagatgacctgtgtatcagggagaaataggcaataaattgagatccagcagggatttgaatcatggatttgaatcaggggcagccttcgaaagaactgtggagaatatactcagatttaaaacataagattggaatttttggcagagaactaacaactgtacaaaaaaggaaccaaatggaaatcctagaactgaaagatgcaattaaccgatgttgagaaatagccaacatctattgaacacttcccatgtggacagctgtgctaaacactttacaggcatcaacataagatgtgtccccttacagcagtgcagtgtccctcctaagacatggacagcctggtttccctatctctctgcttcatcaaaacccctttacgtggggcttagacactcctgttgtctctagtgtctagtagcacagggctcagcacatggaagccactagatacaatttgatgaccaggacctccgatgaaagccatgggtgctgattgggaaggcattgtcttttatgtgctatggtcttaaagcttcatccaggaagcagaactcggggggtgctgaggacccagaaccgagaataagattagtcagagatttcctgtgggcagaaatcataaggacgccaactgtttgggtgagataagacgaaaccaagagtggacttgtggccagaagcgtgaggaagagggagagagcttcccttgtcccctttcttcctctccctaagccacagtgattgacagccccccccctttggagtcagagcaggcttgagactggactgggaaaggagggtgggtcaggatacagagcaggaaggctgggagtgcagggcaggagcaaggggctggggcattcattgtgcctgatctctcccactttacctggggtaaagaagcatatgcaaaagccacggtgtgagtatttcccaagtgccagggtcagggcatgattcatcacgtgcagcatttcattcaatccttatagtaaccgatgatgtggcttctattattagctctatcagataatgaaactgagaccaagacaggctctgcacattgtgtggggtaatgacacagggggattcagacctagactccataactcctgccccagggaccacccccaccctcaccctgtgcatgtcgacaaaggacagactgggccacttctcaggacacagcggggaaatgacacagagcagggaggttccaggagccccgagcgtcttttctccaggagaatactctctgaattcagactggggtcagagaaacatttacccaggagccgcagtgtgggtggggctttttacttgaaacgctgtctgaaggcagtggccaggatggaactctccaccctaccttggcaagccacttctcttctgcaatctgtaaggacattgttgagagaattatggtcttccaattccggagggttgaagaaagacaaataggagagaacctatcatagtcaggtgctagctgccttctctttcagagagtgtgagaataaagtgatacacttgattattagcaaatactttggaaattttaaacgctaatattcaacacactctggaagaggcaaataagtagacaggttcatatacatcatctccttcagctagtcctcacaaaaacaaacaaatgaataaacaaaattcttctttggccctcataggaagacactgtttcttgaacgtgtttcaaaaaggatgggtgactcactcaaggtcacactgtttatgaggacagtacaggaatacagacatgccattttgcctgaaaaaatccatcacccagggaggtgacacaattttgcagaaatgttctatttcctctgaaggatacattctttaaacctttgggaaattcattcatagtcttcctcctttgaaggattaactctctggacacaaagtgtttgattctgatttgttggttggaagatgtgttggttgagagaaagattctgatttgttggttgaaaatagactcatcaagatcaactgctgtagtagtaaatattttgacattttgtctgtattcctgtgctgccctcacaagctgcatcaccttgagtgagtcattcatacttttttgtttgtttttgttttggagatggagtcttactctgttgcctaggctggagtgcggtggcgtgatcttggctcactgcgacctccatctcctgggttcaagtgatcctcctgcctcagcctcccgagtagctgggattacaggcacatgccaccatccctgctaatttttgcattttcagtagagacggagtttcaccatgttggtcaggttggtcttgaactcctgacctcaggtgatccgcccacctcagcctccccaagtgctgggattacaggtgtgagccaccgtgcccagcccagccatcatttttgaaacacgtttgagaaatagtgtcttcctttgagggccaaggagacattttttttgtttatttgtttgtttttgtgaggactagctgaagggggtgatgtatattaacctgcctacttatttgcctcttcccagagtgtgatgaatattagggtttaaagtttctgaagcatttgttaataaagcccggggctggaggtcagaagacctggatttctctgcatacttttgccatcagcaagctgtgtgaccttggacagatcccttttttgtctaaatctttctgagtcttcttgaaaacaatgccaggttgggacaggatgattgccaagctcccgtccagctctaaaacactgcaacgtatgcttctgcaccagcactgtccatcctgtagatcatgcagaaattctcttcaactttttcctacccataaaataggagcatgcttacctttttcctaatgttccaggccccgggtctagaatattgtaagtaaggaagttaatgtgtatcagagcccattatgggccagaagttctcctcttccttcctacacctgcttcctccctccctccctccctctttcccttccttccttccatccatttgtgaagaagacatgatcaccctcattctgagagtgaagagacagaggctcaactaatgaaatgatttgttcaaggtcacacgggtggcacaaggcaagtggcagaggttgaatttagacccattcctgtccaaatgctgagtttatgtcatcgtcccgagaccataactttaaagatgtaagatagtgggaaaagagttgatttcaaagcacctctcagaaggactcactttacatcaggggtcagcagactcaggccaaatccggtccattccccgcttttgcaaagaaagttgtagtggaacacagctaggcttattgatttatggattgccaacgtccttttgtgaaacagacagctgagctgagtaatcgtggcgcacaaaacctaaaatatttactatctcgtcctttacagaatgtttgccaatctatggtccggagtccaaggctgtccatttttcaaagaacacaaagtgacatgagactgtcccatgtgcagggagccctatcattttattatgaaaaaacggcctttctgctcaaatctgttttttaaaaagtcaacaaacagactctgggtacctgtcaggaacagtagggagtttggtttccattgtgctcttcttcccaggaactcaatgaaggggaaatagaaatcttaattttggggaaattgcacaggggaaaaaggggagggaatcagttacaacactccattgcgacacttagtggggttgaaagtgacaacagcaagggtttctctttttggaaatgcgaggagggtatttccgcttctcgcagtggggcagggtggcagacgcctagcttgggtgagtgactatttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcaggtaaggctaccccaaggaggagaaggtgagggtggatcagctggagactggaaacatatcacagctgccaggggctgccaggccccagagggcctgagaactgggtttgggctggagaggatgtccattattcaagaaagaggctgttacatgcatgggcttcaggacttgtgtttcaaaatatcccagatgtggatagtgcgaccggagggctgtcttactttcccagagactcaggaacccagtgagtaatagatgcatgccaaggagtgggactgcgattcaggcctagttgaatgtgctgacagagaagcagagaggggcaccaggggcacagcccgaaggcccagactgatatgggcaaggcctgtctgtgctgacatgtcggagggtcccactctccagggaccttggtttccccgtctgtgacatctgtgacatgagagtcacgataactccttgtgtgccttacagggttgttgtgaaaattaaatgcacagataatagcgtaacagtattccgtgcattgtaaagagcctgaaaaccattatgatttgaaaatggaatcggctttgtgagaccatcactattgtaaagatgtgatgctgatagaaatgacaggactgcttgtgcatgccctctgcagtgtgacattccagcagtgaaatcatgttggggtgacttctcccccactctgacctttatgtttgtctgggccgaggctgcaagtcgggctctgtgggtgtatgagtgacaagtctctcccttccagatatggggactgtctgcttccctaggttgcctctccctgctctgatcagctagaagctccaggagatcctcctggaggccccagcaggtgatgtttatccctccagactgaggctaaatctagaaactaggataatcacaaacaggccaatgctgccatatgcaaagcactttggtttgcctggccacccctcgtcgagcatgtgggctcttcagagccacctgatgaggtgggtacagttagccacacttcacaggtgaagaggtgaggcacaggtcccaggtcaggctggccagagctctgtttattacgtctcacagctttgagtcctgctctcaaccagagaggccctttaccaagaagaaaggattgggacccagaatcaggtcactggctgaggtagagaggaagccgggttgttcccaagggtagctgctcctgcaggactctgagcaggtcaccagctaatggaggaaaggctctagggaaagacccttctggtctcagactcagagcgagttagctgcaaggtgttccgtctcttgaaacttctacctaggtgctatggtagccactagtctcaggtggctatttaaatttatacttaaatgaatgaaaatagaagaaaatttaaaatccagacccttggtcacactatccacatttaaagaggtcaatagccacatgtggttagtggccaccctattgggcagtgcagctacagaacatttttgcatcccagaaagttcttttggatgttgctgctctacagcatgctttgctgaaacagaagtgccttccctgggaatctcagatgggaagcaagtaaggaggggagtcaaatgtgggctcactgctcaccagctgtgagggttgggcctgcctcttaaccattgtcagcctcagtcttctcatccatgcatgccgtgggtatactaaaatactatacccctggaagagctggatgcaaatttgacaagttctgggggacacaggaaggtgccaagcacaaggctgggcacatggtggctgtgcactacagctgagtccttttccttttcagaatctgggatgttaaccagaagaccttctatctgaggaacaaccaactagttgctggatacttgcaaggaccaaatgtcaatttagaaggtgagtggttgccaggaaagccaatgtatgtgggcatcacgtcactttgcccgtctgtctgcagcagcatggcctgcctgcacaaaccctaggtgcaatgtcctaatccttgttgggtctttgtattcaagtttgaagctgggagggcctggctactgaagggcacatatgagggcagcctgaagagggtgtggagaggtagagtctaggtcagaggtcagtgcctataggcacagtggtcccagggccacagctgggaagggcaaataccagaaggcaaggttgaccattcccttcctcaagtgcctattaaggctccatgttcctatgttgttcaaaccctaactcaatcccaaattaatccaccatgtataaggttgagctatgtctcttattcctggacaccatactcagccatattctggtccacacattaaacaagctggatgaccttgaagaagcttcacccactctgttcctcagctttcccttcagtgggatgatatcaactggacaacaggatgtgcgattcttttagttccagccttccaggatgttttcactcccctgtttgttgttgtaggatggtattacctccaccttcccaccttccctatgccctggttctgtctcctgtgcctcgctctgaaagtggatgagacctacaattcctgtcctggtagttctcctaatgaacacactgaagcacgaggaagctgagatttttgttgctacatgagagcatggaggcctcttagggagagaggaggttcagagactcctaggctcctgtggagccccactcatggccttgttcattttccctgcccctcagcaacactcctattgacctggagcacaggtatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcttggtttaatcttctatttacctgcagaccaggaagatgagacctctctgcccttctgacctcgggattttagttttgtggggaccaggggagatagaaaaatacccggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggtaaaaacatgctttggatctcaaatcaccccaaaacccagtggcttgaaacaaccaaaattttttcttatgattctgtgggttgaccaggattagctgggtagttctgttccatgtggtggaacatgctggggtcactttggaagctgcattcagcagagtgcctggcttgcgctgggcatccaaggtggtccctcatcctccaggctctctttccatgtgatctctcagtgtttaagagttagttggagcttccttacagcatggcggctgacttccaaaagggattattccaaaaagagcctcaacatgcaggcgcttattatgacttctgcttgcatcatcctattggccaaagccagtcacgtggctaagtctagccccctgtgagaggagactgcataagagtgtgaacaccaggagacacggtcactgggggccaccactgtaaccatctaccacaggacctgaatctctgtgtgctactcccttgctcaagggcccccctacccacgcagacctgctgtcttctagcaaagcccatcctcaggacctttctcttccaatccttattgactcaaattgattagttggtgctccacccagagccctgtgctcctttatctcatgtaatgttaatgggtttcccagccctgggaaaacatggctttgtctcaggggcttgctggatgcaagcttaacctcaatgtgagtggccatactgtggcactgtcccatccctcaccagggacactgttctggagggtgactgcctgttctgtgaggagtggggatggctaggacattgcatggaacacaccaccaccccatcttctcagagctcaaaccctgacagaacaccagctccacaggccttggcttctgctgatggtgccgtgtatttaccagacttagtggtccaaggccagagtggccagatttcccaaagtcaaggtgtgacagtgggacagcctctttgtgtctttgctgtcctaagaaacctgggccaggccaggcgcagtggctcacgcctgtaatcccagcactttgagaagccaaggtgggcagatcacgaggtcaggagtttgagaccagcctggccaacatggtgaaaccctgtctctattaaaaatagaaaacattagacaggtgtggtggtgcatgcctgtaatcccagctactcaggaggctgaggcaggagaatcgcttgaacccaggaggtggaggttgcagtgagccgagattgtgccactgcactccagcctaggcgacagagcaagactccgtctcgggaaaattaattaataaataaataaacctaggtcccagagtcccacagaatggcagacaggagcacctgggggcttttagggtatggcatttcccctgtactaactctgggctgtccagagggccatttcatggcgtggagtggagagggaggcagcacaggacttcctaggcctcagctctcacctgcccatcttttgatttccaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgccta

SEQ ID NO: 4 - DNA, 1001 bp, Artificial sequence
  IL1RN rs9005
aattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccrggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctatattgttgactatgtccttggccttgacaggctttgggtatagagtgctgaggaaactgaaagaccaatgtgtctttcttaccccagaggctggcgcctggcctcttctctgagagttcttttcttccttcagcctcactctccctggataacatgagagcaaatctctctgcaaaaaagatatggggcagcactgtccacaacagcctctgctggaaacaacccaagcacccatcacagaatgaattagtacatcatgtatctgcacacaacacagtgctccttggcaaagaaaatgaatgaattacagccagctgca

SEQ ID NO: 5 - DNA, 1001 bp, Artificial sequence
  SNP IL1RN rs315952
tcacgaggtcaggagtttgagaccagcctggccaacatggtgaaaccctgtctctattaaaaatagaaaacattagacaggtgtggtggtgcatgcctgtaatcccagctactcaggaggctgaggcaggagaatcgcttgaacccaggaggtggaggttgcagtgagccgagattgtgccactgcactccagcctaggcgacagagcaagactccgtctcgggaaaattaattaataaataaataaacctaggtcccagagtcccacagaatggcagacaggagcacctgggggcttttagggtatggcatttcccctgtactaactctgggctgtccagagggccatttcatggcgtggagtggagagggaggcagcacaggacttcctaggcctcagctctcacctgcccatcttttgatttccaggcagttaacatcactgacctgagcgagaacagaaagcaggacaagcgcttcgccttcatccgctcagacagyggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatc

SEQ ID NO: 6 - DNA, 52 bp, artificial sequence
  Specific locus- IL1RN rs9005
ctgtgcctctgcctgtctcccccaccnggctgggagctctgcagagcaggaa

SEQ ID NO: 7 - DNA, 52 bp, Artificial Sequence
  Specific locus - IL1RN rs315952
cgcttcgccttcatccgctcagacagnggccccaccaccagttttgagtctg

SEQ ID NO: 8 - DNA, 25 bp, Artificial sequence
  rs315952 primer 1
gcttcgccttcatccgctcagacag

SEQ ID NO: 9 - DNA, 25 bp, Artificial sequence
  rs315952 primer 2
ggccccaccaccagttttgagtctg

SEQ ID NO: 10 - DNA, 25 bp, Artificial Sequence
  rs9005 primer 1
tgtgcctctgcctgtctcccccacc

SEQ ID NO: 11 - DNA, 25 bp, Artificial Sequence
  rs9005 primer 2
ggctgggagctctgcagagcaggaa